(19) United States
(12) Patent Application Publication
(10) Pub. No.: US 2025/0061983 A1
(43) Pub. Date: Feb. 20, 2025

(54) SYSTEMS, METHODS, AND DEVICES FOR A CANCER PATHOLOGY LABORATORY INFORMATION SYSTEM (LIS)

(71) Applicant: Pathology Watch Inc., Salt Lake City, UT (US)

(72) Inventors: Daniel Lambert, Salt Lake City, UT (US); Evan Cancelliere, Toronto (CA)

(73) Assignee: Pathology Watch Inc., Salt Lake City, UT (US)

(21) Appl. No.: 18/452,528

(22) Filed: Aug. 19, 2023

(51) Int. Cl. G16H 10/40; G06F 9/451; G16H 10/60; G16H 40/63

(52) U.S. Cl. CPC G16H 10/40; G06F 9/451; G16H 10/60; G16H 40/63

(57) ABSTRACT

Systems, methods, and devices for a cancer pathology laboratory information system (LIS) are described herein. Features and solutions described may enhance the efficiency and accuracy with which tissue samples are recorded, processed, and tracked for pathology. For example, a dynamic technician interface may be provided to minimize the quantity of inputs and/or potential errors that may be made by a technician. Also provided are solutions for monitoring, analyzing, and correcting improper or inefficient technician conduct. Many other features and examples are described herein.

### FIELD

This disclosure relates to electronic systems and devices for cancer related pathology.

### BACKGROUND

Laboratories for cancer related pathology typically determine pathology for patients based on physical tissue samples. Often, a tissue sample is received, cut into thin slices, put onto slides, stained, and inspected under a microscope by a physician. Many laboratories perform these operations using physical and manual processes.

### BRIEF DESCRIPTION OF THE DRAWINGS

The present disclosure will be readily understood and enabled by the detailed description and accompanying figures of the drawings. Like reference numerals may designate like features and structural elements. Figures and corresponding descriptions are provided as non-limiting examples of aspects, implementations, etc., of the present disclosure, and references to “an” or “one” aspect, implementation, etc., may not necessarily refer to the same aspect, implementation, etc., and may mean at least one, one or more, etc.

FIG. 1 is a diagram of an example network environment according to one or more implementations described herein.

FIG. 2 is a diagram of a technician station according to one or more implementations described herein.

FIG. 3 is a diagram of an example of a process for generating an interface at a technician station and maintaining accurate sample records according to one or more implementations described herein.

FIG. 4 is a diagram of an example of a requisition form 400 according to one or more implementations described herein.

FIG. 5 is a diagram of an example of a requisition form, a case QR code, and corresponding specimens according to one or more implementations described herein.

FIG. 6 is a diagram of an example of a specimen and specimen label according to one or more implementations described herein.

FIG. 7 is a diagram of an example of using QR codes to logically associate a case with multiple biopsy samples according to one or more implementations described herein.

FIG. 8 is a diagram of an example of records created by an LIS according to one or more implementations described herein.

FIG. 9 is a diagram of an example of sample procedure types and interfaces according to one or more implementations described herein.

FIGS. 10-18 are diagrams of examples of technician station interfaces according to one or more implementations described herein.

FIG. 19 is a diagram of an example 1900 of sample slides of a biopsy sample or specimen according to one or more implementations described herein.

FIG. 20 is a diagram of an example of a sample slide according to one or more implementations described herein.

FIG. 21 is a diagram of an example of using QR codes to logically associate a biopsy sample with biopsy sample slices according to one or more implementations described herein.

FIG. 22 is a diagram of an example of an association of QR codes between a case, biopsy samples, and sample slices according to one or more implementations described herein.

FIG. 23 is a diagram of an example of a technician terminal according to one or more implementations described herein.

FIG. 24 is a diagram of an example laboratory information system (LIS) server according to one or more implementations described herein.

FIG. 25 is a diagram of an example of a process for dynamically ensuring technician input accuracy according to one or more implementations described herein.

FIG. 26 is a diagram of an example of a neural network (NN) according to one or more implementations described herein.

FIG. 27 is a diagram of an example of components of a device according to one or more implementations described herein.

### DETAILED DESCRIPTION

The following detailed description refers to the accompanying drawings. Like reference numbers in different drawings may identify the same or similar features, elements, operations, etc. Additionally, the present disclosure is not limited to the following description as other implementations may be utilized, and structural or logical changes made, without departing from the scope of the present disclosure.

Pathology laboratories may operate using physical and manual processes. For example, a physical tissue sample may be received at the laboratory, cut into thin slices, put onto slides, stained, and inspected under a microscope by a physician. In recent years, digital pathology (e.g., an ability to create digital versions of slides by scanning physical slide) has introduced the ability to develop automated processes, design software tools, train and implement neural networks (NNs), integrate previously disconnected communication and record keeping systems, etc., to enhance the level of quality, accuracy, efficiency, and effectiveness which with a laboratory, or group of laboratories, may operate. One or more of these features and their corresponding advantages are described herein as a laboratory information system (LIS). The increased automation, quality and effectiveness of such laboratories will, in turn, have secondary benefits of enabling pathology laboratories to provide higher quality services to more patients at lower costs than laboratories that fail to implement the systems, devices, and techniques described herein.

A laboratory may include multiple technician stations 210 designed to enable the preparation, processing, and examination of a biopsy sample and medical data for determining pathology. Examples of such activities may include accessioning, grossing, processing, cutting and embedding, staining, and digitizing. Accessioning may include inputting data about a patient and corresponding sample into the LIS. For example, when a tissue sample arrives at a laboratory, the sample may include a label, text, a barcode, a quick response (QR) code, and/or other types of information that may be used to enter information about the sample and patient into the LIS. Grossing may include measuring and entering the physical dimensions of the sample into the system. Processing may include dehydrating the physical sample and removing sample features identified as irrelevant, unnecessary, or of lesser importance. Cutting and embedding may include putting the biopsy sample in wax and cutting the sample into thin slices. Staining may include making the color of tumorous tissue standout visually relative to non-tumorous tissue. Digitizing may include scanning each sample slide to create a digital copy of each slide. Proper execution of each of these procedures is critical to maintain the accuracy of the sample and patient information input into the LIS and the quality of the sample slides.

Systems, methods, and devices for cancer pathology LIS are described herein. The technics described herein may be applied to melanoma, basal cell, squamous cell, Merkel cell, Seborrheic Keratosis, Actinic Keratosis, and Verruca Vulgaris pathology, cysts, and more. The features and solutions described with reference to the Figures below may enhance the efficiency and accuracy with which tissue samples are recorded, processed, and tracked for pathology. For example, a dynamic technician interface may be provided to minimize the quantity of inputs and/or potential errors that may be made by a technician. Also provided are solutions for monitoring, analyzing, and correcting improper or inefficient technician conduct. Many other features and examples are also described herein.

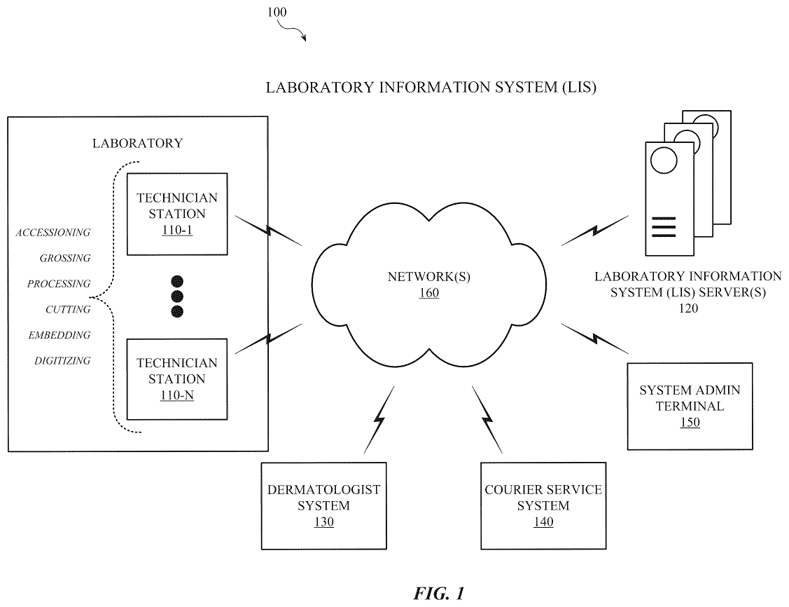

FIG. 1 is a diagram of an example environment 100 in which systems and/or methods, described herein, may be implemented. As depicted, environment 100 may include a laboratory with technician stations 110-1, . . . 110-N (where N is greater than or equal to 1 and collectively referred to as “terminals 110”), LIS servers 120, dermatologist system 130, courier service system 140, system admin terminal 150, and network 160. The number of devices and/or network, illustrated in FIG. 1, is provided for explanatory purposes only. In practice, there may be additional devices and/or networks, fewer devices and/or networks, different devices and/or networks, or differently arranged devices and/or networks than illustrated in FIG. 1. Devices of environment 100 may interconnect via wired connections, wireless connections, or a combination of wired and wireless connections. Also, one or more of the devices of environment 100 may perform one or more functions described as being performed by one or more other devices of environment 100.

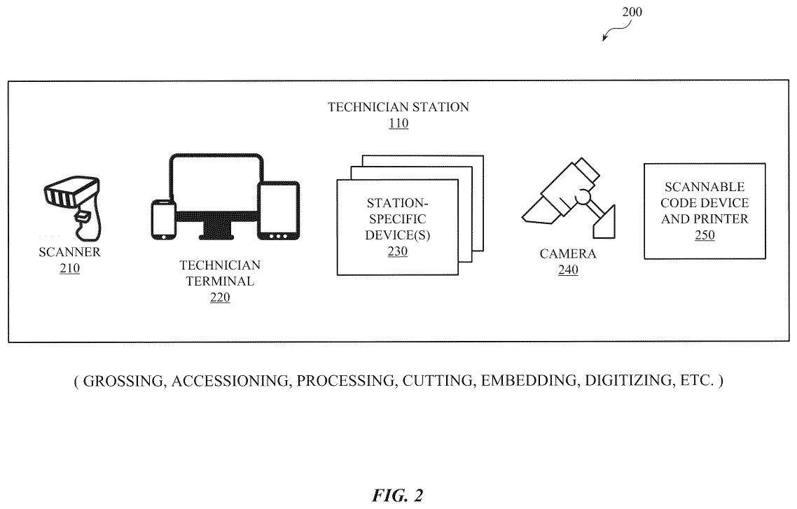

Technician station 110 may include a location within a laboratory for processing tissue samples and entering tissue sample and patient information into a LIS. As mentioned above, such activities may include accessioning, grossing, processing, cutting and embedding, staining, and digitizing. FIG. 2 is a diagram of an example technician station 110 according to one or more implementations described herein. As shown, technician station 110 may include scanner device 210, technician terminal 220, one or more station-specific devices 230, and a camera 240. Scanner device 210 may be used to scan a barcode, QR code, and/or other information from a label of a tissue sample and a technician badge. This may cause a record to be created of the tissue sample and technician being located at the technician station at a corresponding date and time. As described herein, it may also cause a sequence of interface, used by the technician to enter additional information via technician terminal 220, to be automatically populated by the LIS.

Station-specific devices 230 may include one or more devices configured to enable a technician to perform a procedure designated to the station, such as removing unnecessary tissue from a tissue sample, cutting the tissue sample into thin slices, placing the sample slices on separate slides, staining the slices, etc. When a tissue sample and technician badge are scanned by scanning device 210, camera 240 may automatically begin recording a video of what happens at technician station 110. In some implementations, this may enable evaluation of the technician and technician station to assess technician efficiency, technician accuracy, tissue processing quality, analysis and review in the event of an error, etc. In some implementations, the camera may be high-resolution enough to capture image data from which the LIS may extract patient data, determine metadata about the tissue sample, and analyze and assess the recorded session.

Scannable code device and printer 250 may include any type of computing device and/or printer capable of generating a barcode, QR code, or another type of scannable image or pattern. Scannable code device and printer 250 may also be capable of printing a label with the scannable image or pattern, such that the label may be placed on a case intake or registration form, a biopsy sample bag or cannister, and/or biopsy slide. In some implementations, some or all of the functions of scannable code device and printer 250 may be performed by technician terminal 220.

Referring to FIG. 1, LIS servers 120 may include one or more servers or other types of computing devices capable of gathering, processing, searching for, storing, and/or communicating information as described herein. LIS servers 120 may communicate with scanner device 210, technician terminal 220, dermatologist system 130, courier service system 140, and/or system admin terminal 150 via networks 260. In some implementations, LIS servers 120 may perform, and/or provide a platform for performing, one or more of the processes described herein. For example, LIS servers 120 may create and update records based on scan inputs originating from scanner 210, may enable or facilitate interfaces to be provided to technician terminal 220, create video records created by inputs from camera 240, tack tissue samples and slides transported by courier services, and support the application of neural networks (NNs) toward technician practices, instances of handling tissue samples, and providing analysis and corrective feedback, etc.

Dermatologist system 130 may include any type of wired or wireless user device capable of communicating with LIS servers 120 via network 160. Dermatologist system 130 may include a smartphone, tablet computer, laptop computer, desktop computer, server, or another type of user device capable of enabling a user, operator, administrator, or developer to interact with LIS servers 120. Dermatologist system 130 may enable a dermatologist or other type of physician (and/or assistant) to interact with LIS servers 120 via network 160. For example, dermatologist system 130 may enable a physician to search for the location of a particular sample, review the handling of a particular sample, review the recorded actions of a technician, etc.

Courier service system 140 may include any type of wired or wireless user device capable of communicating with LIS servers 120 via network 160. Courier service system 140 may include a smartphone, tablet computer, laptop computer, desktop computer, server, or another type of user device capable of enabling a user, operator, administrator, or developer to interact with LIS servers 120. Courier service system 140 may be owned and operated by an organization or company that transports tissue samples, and/or slides of tissue samples, from one geographic location to another. Courier service system 140 may communicate with LIS servers 120 to convey the prior location, current location, and/or future location, including scheduling information, of tissue samples and/or slides of tissue samples. LIS servers 120 may extract information from courier service system 140 via application interface (API) or web scraping.

System administration terminal 150 may include any type of wired or wireless user device capable of communicating with LIS servers 120 via network 160. System administration terminal 150 may include a smartphone, tablet computer, laptop computer, desktop computer, or another type of user device capable of enabling a user, operator, administrator, or developer to interact with LIS servers 120. Additionally, or alternatively, system administration terminal 150 may be directly connected to LIS servers 120. System administration terminal 150 may be configured to track samples, barcodes of samples, and/or one or more other types of sample identifiers through the laboratory processing. System administration terminal 150 may be configured to support, perform, or enable the management of a pathology image.

Network 160 may include a single network or multiple networks capable of enabling a connection between the devices of FIG. 2. Network 160 may include one or more wired and/or wireless networks. For example, network 160 may include a Bluetooth® network, a Wi-Fi network, or a cellular network, the Public Land Mobile Network (PLMN), and/or a second generation (2G) network, a third generation (3G) network, a fourth generation (4G) network, a fifth generation (5G) network, a sixth generation (6G) network and/or another type of network. Additionally, or alternatively, network 160 may include a wide area network (WAN), a metropolitan area network (MAN), an ad hoc network, an intranet, the Internet, a virtual network (e.g., a virtual private network (VPN)), a telephone network (e.g., a Public Switched Telephone Network (PSTN)), a Voice over IP (VOIP) network, and/or a combination of these or other types of networks.

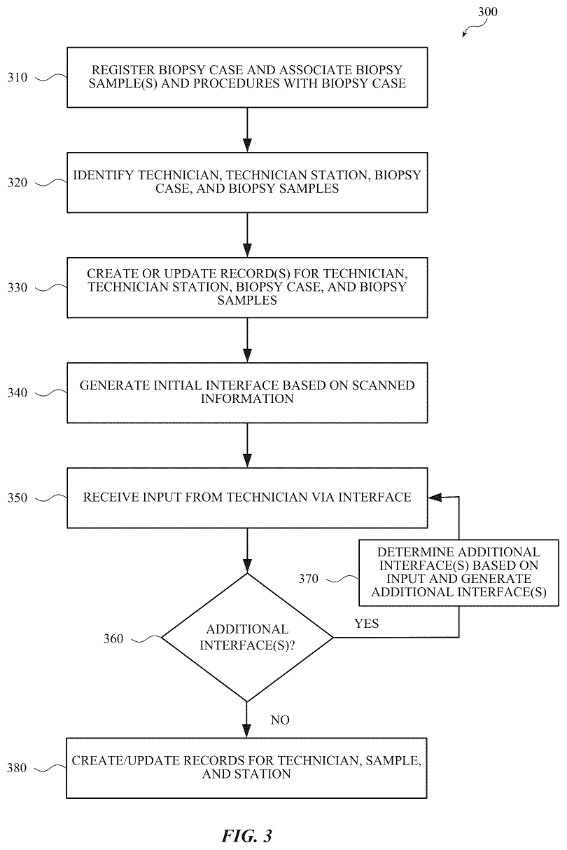

FIG. 3 is a diagram of an example of a process 300 for generating an interface at a technician station and maintaining accurate sample records according to one or more implementations described herein. Process 300 may be implemented by LIS servers 120. In some implementations, some or all of process 300 may be performed by one or more other systems or devices, including one or more of the devices of FIG. 1. Additionally, process 300 may include one or more fewer, additional, differently ordered and/or arranged operations than those shown in FIG. 3, including other processes and/or operations discussed herein. For example, process 300 may include operations preceding, performed in parallel with, and/or following one or more of the depicted operations. Furthermore, some or all of the operations of process 300 may be performed independently, successively, simultaneously, etc., of one or more of the other operations of process 300. As such, the techniques described herein are not limited to the number, sequence, arrangement, timing, etc., of the operations or process depicted in FIG. 3.

Generally, some or all of process 300 may be implemented at one or more technician stations 110 in a laboratory environment. As such, some or all of process 300 may be implemented within the context of accessioning, grossing, processing, cutting and embedding, staining, and digitizing. As mentioned above, accessioning may include inputting data about a patient and corresponding sample into the LIS. For example, when a tissue sample arrives at a laboratory, the sample may include a label, text, a barcode, a QR code, and/or other types of information that may be used to enter information about the sample and patient into the LIS. Grossing may include measuring and entering the physical dimensions of the sample into the system. Grossing may include automatically measuring and entering physical dimensions, via analysis of an image or video feed. Processing may include dehydrating the physical sample and removing sample features identified as irrelevant, unnecessary, or of lesser importance. Cutting and embedding may include putting the cancer sample in wax and cutting the sample into thin slices. Staining may include making the color of tumorous tissue standout visually relative to non-tumorous tissue. Digitizing may include scanning each sample slide to create a digital copy of each slide. Proper execution of each of these procedures is critical to maintain the accuracy of the sample and patient information input into the LIS and the quality of the sample slides.

In some laboratories, a different technician station 110 may be used for each of accessioning, grossing, processing, cutting and embedding, staining, and digitizing. In some laboratories a different allocation may be used. For example, one technician station 110 may be used for accessing; another for grossing and processing; another for cutting and embedding, and another for staining and digitizing. In yet other laboratories, accessioning, grossing, processing, cutting and embedding, staining, and digitizing may all be completed at a single technician station 110. As such, some or all of process 300 may be implemented at a technician station depending on the situation. In other laboratories, samples may be sent outside of the facility for additional work, or the sequence order may be different. The sequence of accessing, grossing, processing, cutting, and embedding may be different.

As shown, process 300 may include registering a biopsy case and associating the biopsy case with biopsy samples and procedures (block 310). For example, when a biopsy sample is received at a laboratory, a technician may create a record of the biopsy case. This may include entering information and creating an electronic record of the biopsy case in the LIS. The information assigned or associated with the electronic record may include a case identifier to uniquely identifies the case, patent information, specimens associated with the biopsy case, and so on. In some implementations, some or all of this information may already be in the LIS, and the technician may access the information using a case identifier for the biopsy case. The technician may also, or alternatively, create a scannable code, such as a barcode or QR code for the biopsy case and print a requisition form (or case form) that includes information about the biopsy case.

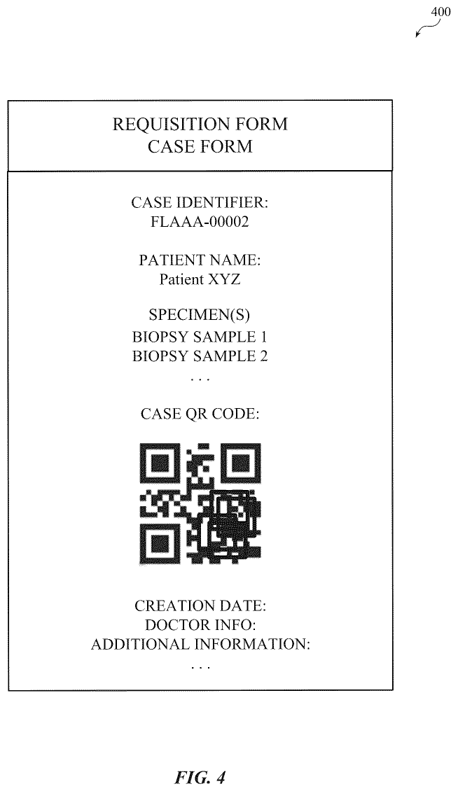

FIG. 4 is a diagram of an example of a requisition form 400 according to one or more implementations described herein. As shown, requisition form 400 may include a case identifier, patient name, and identifiers for specimens associated with the case (e.g., biopsy sample 1, biopsy sample 2, etc.). Requisition form 400 may also include a case QR code that references electronic records associated with the case, doctor information, the date the case was received or requisitioned, and/or one or more additional types of information. As samples are processed at technician station within the laboratory, sent to other laboratories, sent to doctor offices, etc., requisition form 400 may accompany the samples to enable identification, tracking, and proper inventorying of the samples.

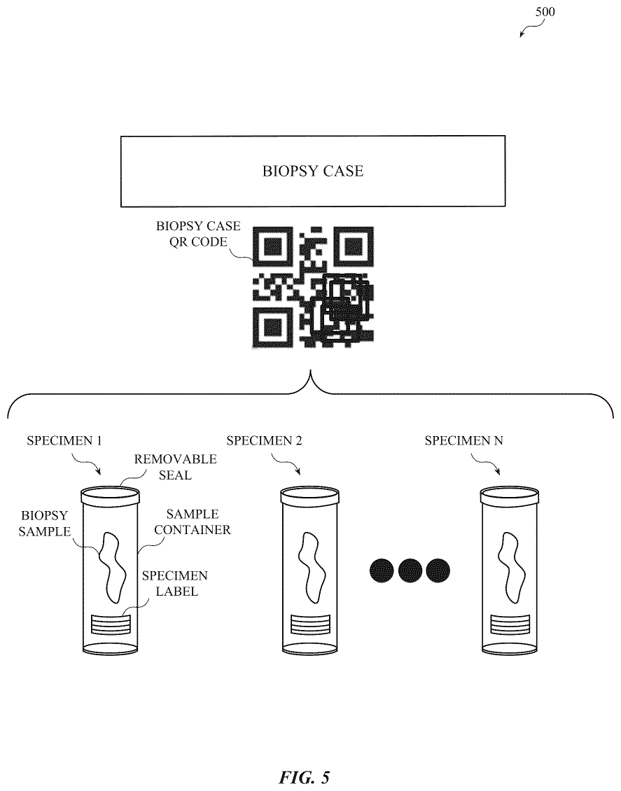

FIG. 5 is a diagram of an example 500 of a requisition form, a case QR code, and corresponding specimens according to one or more implementations described herein. The requisition form may include information about the case, including a case QR code. Each specimen may include a biopsy sample within a sample container with a removable seal. A technician may operate technician station 110 to create a specimen label for each specimen and to create an electronic record that associates the specimens with the case and case QR code.

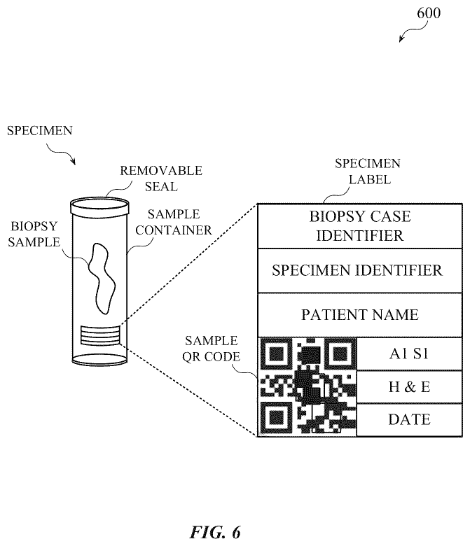

FIG. 6 is a diagram of an example 600 of a specimen and specimen label according to one or more implementations described herein. As shown, a specimen label may include one or more types of information about the specimen, such as a case identifier that uniquely identifies the case, a specimen identifier that uniquely identifies the specimen (e.g., A1), a slide number (e.g., S1), a patient name, and stain type (e.g., H & E). The specimen label may also include a QR code that may be scanned at technician stations to reference electronic records associated with the case and specimen. When a case and corresponding specimens arrive at the laboratory, a technician may create a specimen QR code and a specimen label to enable identification, tracking, and proper inventorying of the samples.

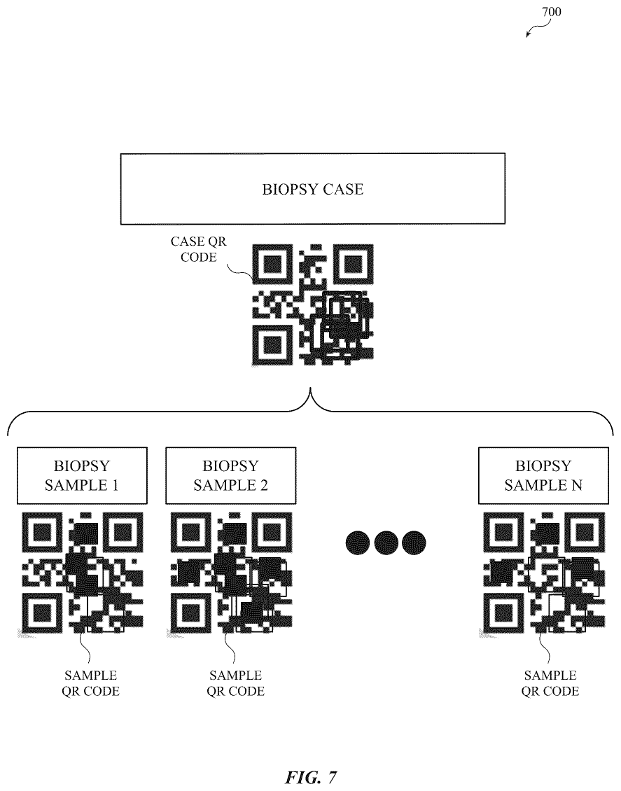

FIG. 7 is a diagram of an example 600 of using QR codes to logically associate a case with multiple biopsy samples according to one or more implementations described herein. As shown, as a case may include multiple physical biopsy samples, the case and each biopsy sample may be assigned a QR code and the QR code of the case may be logically or electronically associated with the QR code of the biopsy samples of the case. In this manner, as a case and biopsy samples go from one technician station 110 to another, from one laboratory to another, and/or to a doctor's office or other locations, electronic records associating the samples via QR codes may be used to access various LIS features such as tracking, record keeping, orderliness, the investigation of errors, and others discussed below with respect to Figs. that follow.

Referring to FIG. 3, process 300 may include identifying a technician, technician station, a biopsy case, and biopsy samples (block 320). For example, a technician may carry an ID badge that includes identification information configured to uniquely identify the technician. Upon arriving at technician station 110, the technician may use scanner 210 to scan the technician's ID badge, and/or the QR code of the biopsy case. The technician may also, or alternatively, scan one or more of the QR codes of biopsy samples of the biopsy case. Technician terminal 220 and/or LIS server 120 may use the scanned information to determine corresponding identifiers for the technician, biopsy case, and/or biopsy samples. Technician terminal 220 and/or LIS server 120 may determine an identity of the technician station based on scanner 210, technician terminal 220, or another device associated with the technical station. The technician terminal 220 may remain locked or secure unless a valid technician ID badge is used.

Process 300 may include creating or updating records for the technician, technician station, biopsy case, and biopsy samples (block 330). For example, in response to scanning QR codes the LIS (e.g., technician terminal 220 and/or LIS servers LIS servers 120) may proceed to create an electronic record of the technician at the technician terminal with the biopsy samples. The record may include additional information as well, such as the time, date, amount of time the technician is at the station and/or working on a particular biopsy case or biopsy sample, etc. In some implementations, camera 240 and appropriate image recognition software may also, or alternatively, be used to register technician at the technician terminal with the biopsy samples. In some implementations, scanning the technician's ID badge, the biopsy case QR code, and/or biopsy sample QR codes, may cause camera 240 at technician station 110 to begin recording. Additionally, when the technician has finished working on one biopsy sample at technician station 111, the technician may scan the QR code of the next biopsy sample prior to starting work on the next biopsy sample. In response, the LIS may cause the prior video recording to be stored as a separate file before initiating a new video recording of the technician working on the recently scanned biopsy sample.

In this manner, the LIS may be configured to automatically create an organized and easily searchable set of video files for each technician, technician station, biopsy case, and each biopsy sample. Doing so may, for example, facilitate the review and resolution of problems, such as when a biopsy sample has been misplaced or mishandled. Doing so may also facilitate recognition of a faulty piece of equipment inside of the lab. Doing so may also facilitate operational goals such as identifying what some technicians may be doing particularly well, ways in which technicians or operations may improve, determining proper work environment goals and expectations, etc.

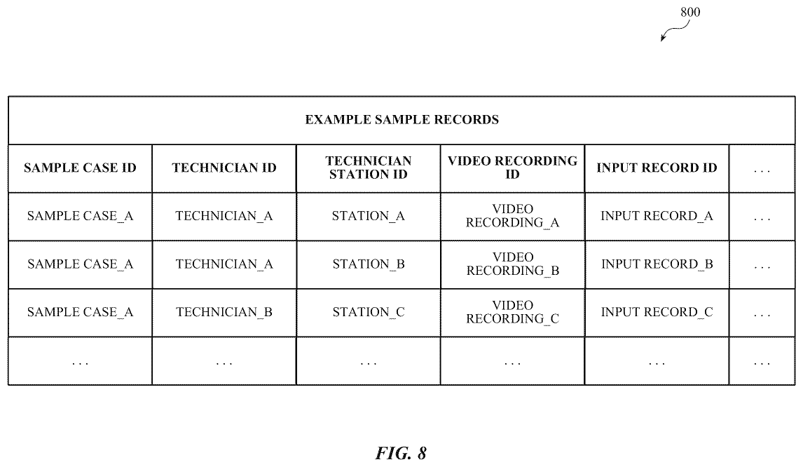

FIG. 8 is a diagram of an example of records 800 created by an LIS according to one or more implementations described herein. As described herein, LIS server 120 may receive various types of information from any one or combination of scanner 210, technician terminal 220, camera 240, and one or more other devices at a laboratory or technician station 110. LIS server 120 may create logical associations between the information and store the information as records. The example of FIG. 8 is provided as a non-limiting example of the techniques described herein. In one or more other implementations example records may include one or more fewer, additional, and/or alternative types of records. Additionally, or alternatively, one or more records may include fewer, additional, or alternative types of information, or an alternative arrangement thereof.

As shown, records 800 include a sample case ID, technician ID, technician station ID, video recording ID, and input record ID. The sample case ID may uniquely identify a case. Technician ID may uniquely identify a technician; technician station ID may uniquely identify a technician station; video recording ID may uniquely identify a video recording made at that station while the sample case was being worked on; and input record ID may uniquely identify records of inputs made by a technician. As such, each record relates to sample case A, and technician A worked on sample case A at technician stations A and B. When sample case A was transferred to technician station C, however, technician B began work on sample case A. In this manner, one or more of the techniques described herein include solutions for efficient, reliable, and easily searchable record creation and management.

Process 300 may include generating an initial interface based on the scanned information (block 340). For example, based on information scanned into the LIS or information input into the LIS by a technician, LIS may generate an initial interface, accessible via technician terminal 220, for processing a biopsy sample at technician station 110. An interface, as described herein, may include a graphical user interface presented via technician terminal 220. The interface may include one or more of a variety of interface features, such as alphanumeric text, graphics, radio buttons, check boxes, lists, options, buttons, prompts, or other features. The LIS may determine an initial interface and subsequent interfaces (referred to collectively as a sequence of interfaces) based on a biopsy sample scanned into the LIS, other information input into the LIS by a technician, physician, and/or code and data already stored by the LIS. The sequence of interfaces may facilitate and support a technician in completing her/his responsibilities at technician station 110.

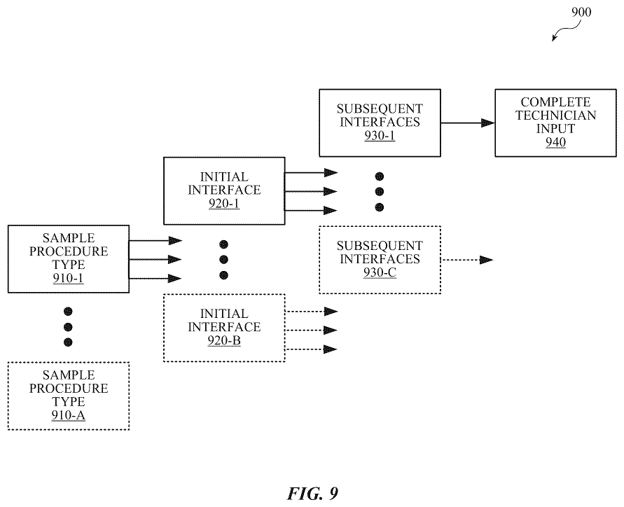

FIG. 9 is a diagram of an example 900 of sample procedure types and interfaces according to one or more implementations described herein. As shown, LIS may include a record of sample procedure types 910-1 through 910-A (where A is greater than 1) for processing certain types of biopsy samples in certain types of ways. Examples of types of biopsy samples may include a shave biopsy, a punch biopsy, an excision, etc., and/or a location of the body where the biopsy was taken. Processing a biopsy sample may include one or more of accessioning, grossing, cutting and embedding, etc., and/or may include inputting information into LIS.

Each sample procedure type 910 may be associated with one or more initial interfaces 920-1 through 920-B (where B is greater than 1), and each initial interface may be associated with one or more subsequent interfaces 930-1 through 930-C (where C is greater than 1). In some implementations, which subsequent interfaces 930 are associated with an initial interface 920 (or a previous interface) may depend on information entered by the technician in one or more preceding interfaces. Completing the sequence of interfaces may be associated with completing the corresponding technician input 940 task. As described herein, the interfaces may assist technicians in performing their duties accurately and efficiently, by for example, prompting technicians to enter certain types of data, ensuring that the data entered is complete, and so on. Examples of biopsy samples, interface sequences, etc., are discussed further below with reference to the Figures.

Referring to FIG. 3, process 300 may include receiving input from a technician via an interface (block 350). For example, technician terminal 220 and/or LIS server 120 may receive information input by a technician. Upon completion of the interface, technician terminal 220 and/or LIS server 120 may determine whether there is an additional interface to be completed relative to the biopsy sample procedure (block 360). Where there is an additional interface to complete (block 360—YES) process 300 may proceed by determining the additional interface based on technician inputs and generating the additional interface (block 370). Process 300 may proceed to receiving input from the technician (block 350).

When there is not an additional interface to complete (block 360—No) process 300 may proceed by creating and/or updating records for the technician, sample, and technician station (block 380). For example, once the technician has completed the sequence of interfaces for a particular biopsy sample (and/or a set of interface sequences for multiple biopsy samples for a case) technician terminal 220 and/or LIS server 120 may cause records stored by the LIS to be updated in accordance with the information input by the technician via the interface sequences. This may include creating and/or updating records such as those described above with reference to FIG. 8 or other/additional types of records.

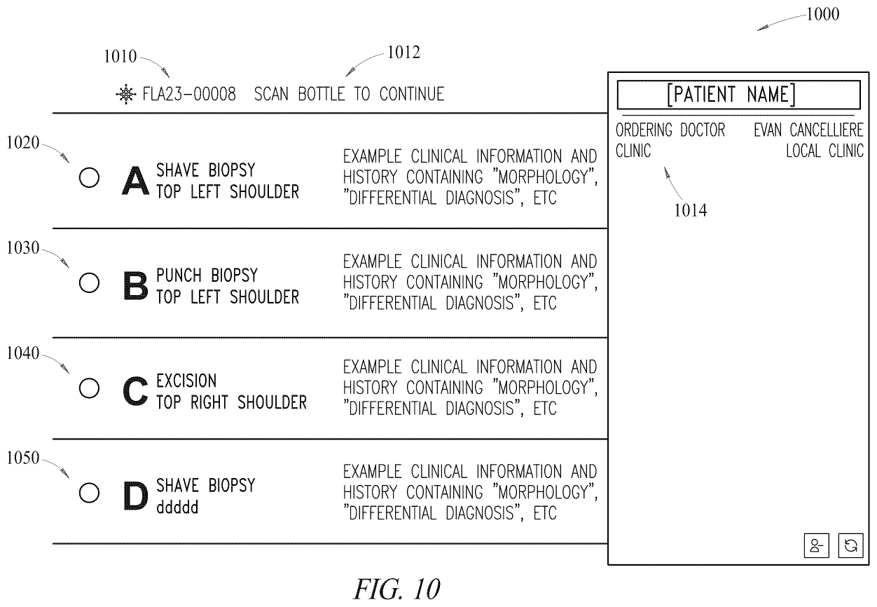

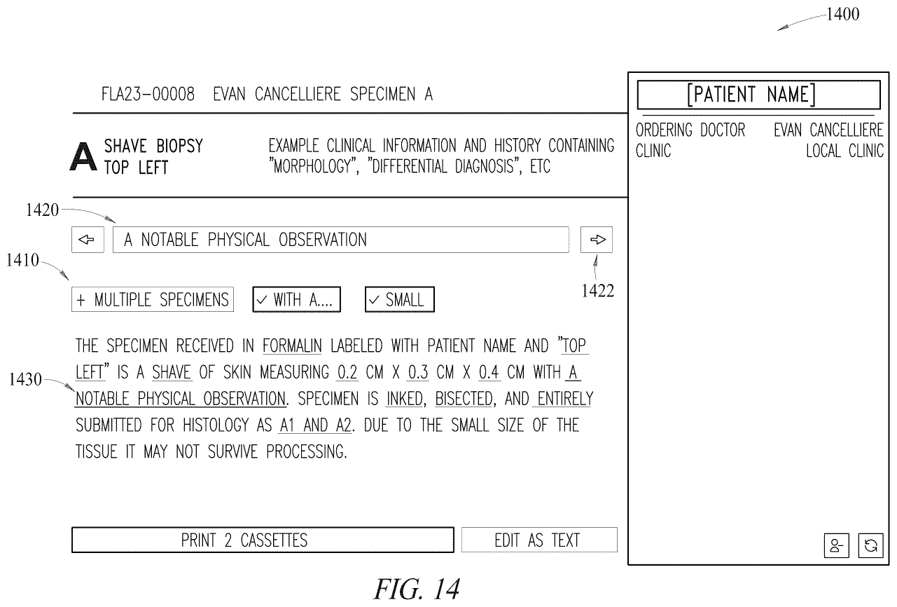

FIGS. 10-18 are diagrams of examples of interfaces 1000-18000 according to one or more implementations described herein. One or more of interfaces 1000-18000, including the features depicted therein, may be viewed as an interface that may be implemented independently. Additionally, or alternatively, some or all of interfaces 1000-18000 may be viewed as a sequence of interfaces corresponding to one or more biopsy sample procedures. Furthermore, interfaces 1000-18000 are provided as non-limiting examples. Interfaces, and sequences of interfaces, may include fewer, additional, alternative, and/or alternatively arranged or configured interfaces than those depicted in FIGS. 10-18.

Referring to FIG. 10, interface 1000 may include a biopsy case number or identifier 1010 and a list of selectable sample biopsies 1020, 13030, 1040, 1050 associated with biopsy case. Each of the sample biopsies may include an identifier A-D, a short description of the biopsy sample (e.g., “shave biopsy top left shoulder”) and additional information. Interface 1000 may also include additional information about the biopsy case, such as an ordering doctor, clinic, technician, etc., 1014. Interface 1000 may also include instructions 1012 on what the technician may do in order to proceed with the interface sequence.

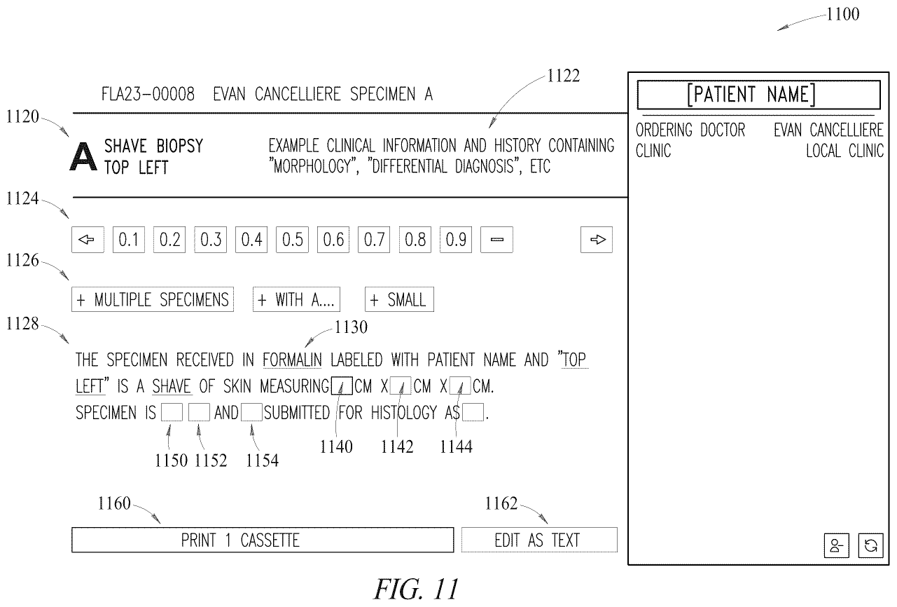

Referring to FIG. 11, interface 1100 may also include information, such as a biopsy case identifier, name of a patient or technician, an ordering doctor, an ordering clinic, a selected biopsy sample or specimen (e.g., Specimen A 1120), etc. Interface 1100 may further include default text 1128 automatically loaded into an interface template that also includes information boxes 1130-1154 that may be filled in by the technician. Interface 1100 may further include selectable options 1124 and 1126 for filling in one or more information boxes 1130-1154. As a technician fills in each box, selectable options 1124 and 1126 may update their content according to the nature of the information that is appropriate for subsequent boxes. Interface 1100 may also include a button 1160 for printing a cassette and/or a button 1162 for manually editing the template text. Printing a cassette may include inking or etching a barcode or numerical sequence or other patient identifier onto the surface of the cassette. The features of interface 1100 may help ensure that technicians efficiently and accurately input clear and relevant information about the biopsy sample, which will be important to further analysis and consideration in developing an appropriate prognosis.

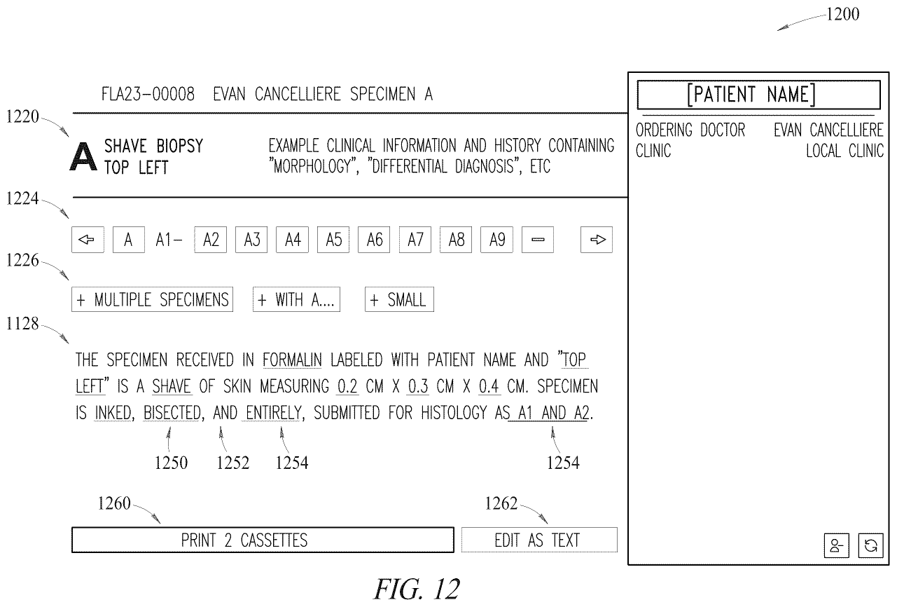

Referring to FIG. 12, interface 1200 may also include information, such as a biopsy case identifier, name of a patient or technician, an ordering doctor, an ordering clinic, a selected biopsy sample or specimen (e.g., Specimen A 1220), etc. Interface 1200 may further include default text 1128 automatically loaded into an interface template and text input by a technician 1250-1254, using selectable options 1224 and 1226. Interface 1200 may also include a button 1260 for printing 2 cassettes and/or a button 1262 for manually editing the template text. Interface 1200 may include an option to print 2 cassettes (instead of 1 cassette as in FIG. 11) because there may be excess additional tissue, or the case requires 2 cassettes because of the nature of the tissue.

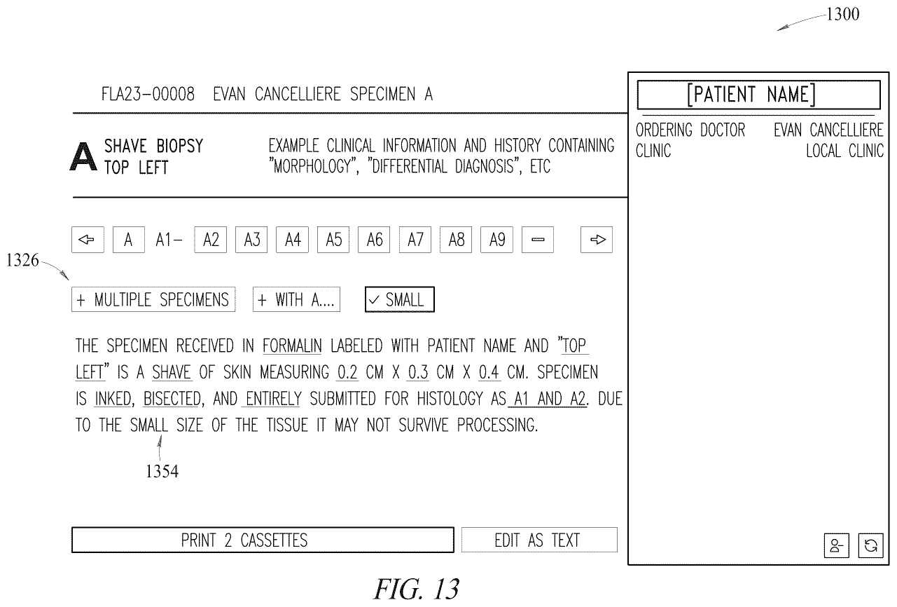

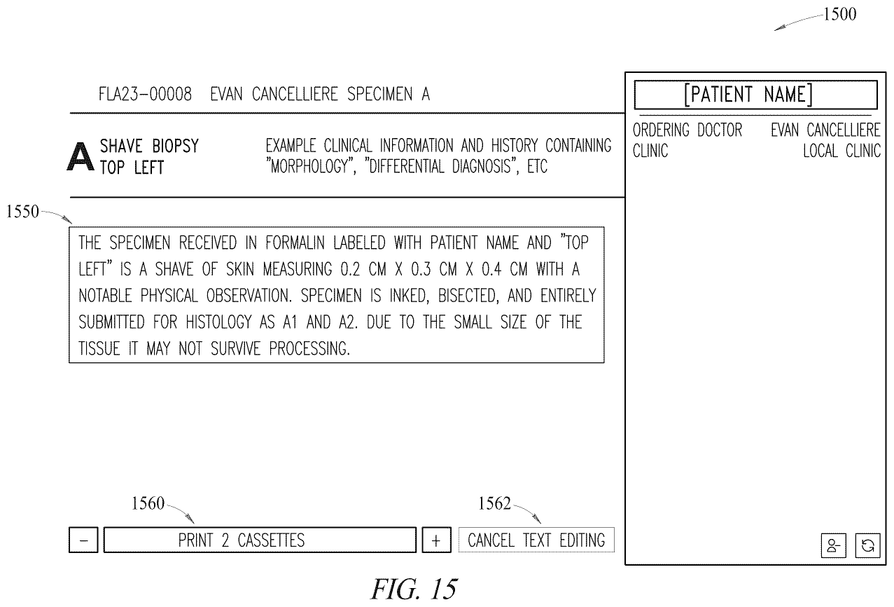

Referring to FIG. 13, interface 1300 includes a scenario in which the biopsy sample may be small, and therefore the technician may select “Small” from the set of preloaded options 1326. As shown, doing so may include a corresponding phrase 1354 within the text of interface 1300. Referring to FIG. 13, interface 1400 includes a scenario where the technician decides to add an additional phrase within the text to be submitted to the LIS. As such, the technician may select the option “with a . . . ” 1410 in interface 1400. This may cause interface 1400 to dynamically create a text box 1420 to enable the technician to type in a particular phrase or scroll through a set of prepared phrases, such as “a notable physical observation,” and select an appropriate phrase. Interface 1300 may then insert the phrase at an appropriate location 1430 within the text. Referring to FIG. 15, when the technician has worked through all of the interfaces and/or interface features, interface 1500 may be presented in completed form 1550 to the technician for final review and approval. Additionally, or alternatively, the technician may edit the final text by pressing the “edit as text” button (see, e.g., interface 1400). Interface 1500 may also include a button to print 2 cassettes and/or a button to cancel text editing.

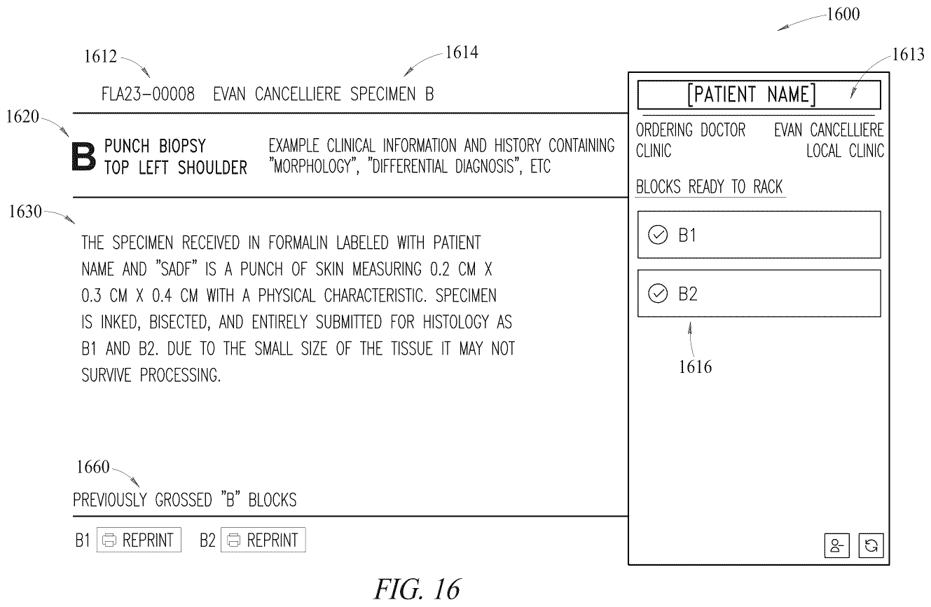

Referring to FIG. 16, interface 1600 may correspond to a selection of Specimen B 1030 of FIG. 10. Similar to interface 1100 of FIG. 11, interface 1600 may include information, such as a biopsy case identifier 1612, name of a patient or technician, an ordering doctor and receiving clinic 1613, a selected biopsy sample or specimen (e.g., Specimen B 1614), etc. Interface 1600 may undergo a process similar to that of the sequence of interfaces for Specimen A depicted in FIGS. 11-15. As such, interface 1600 may include a combination 1630 of default text that is automatically loaded into an interface template and information selected or otherwise inputted by a technician through a series of prompts. Interface 1600 may also include an indication 1660 of previously grossed “B” blocks. Interface 1600 may further include an indication 1616 of which blocks are ready to rack 1616 (e.g., blocks B1 and B2). Racking a block, as described herein, may include placing the block into a holder that contains other blocks from the day. The purpose of the holder, or rack, may including housing other blocks from that day (e.g., in case the blocks need to be found later for a medical or legal reason).

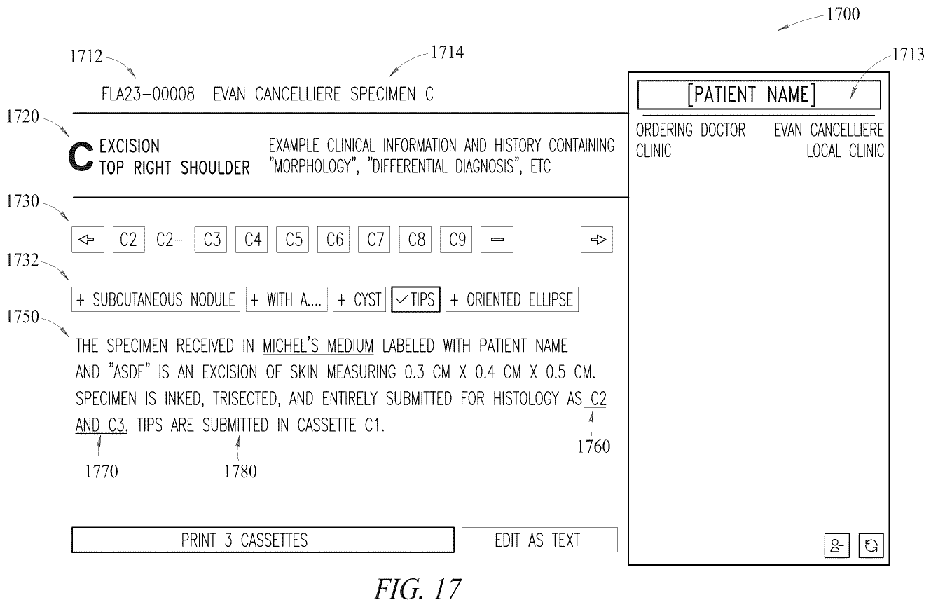

Referring to FIG. 17, interface 1700 may correspond to a selection of Specimen C 1030 of FIG. 10. Similar to interface 1100 of FIG. 11, interface 1700 may include information, such as a biopsy case identifier 1712, name of a patient or technician, an ordering doctor and receiving clinic 1713, a selected biopsy sample or specimen (e.g., Specimen C 1714), etc. Interface 1700 may undergo a process similar to that of the sequence of interfaces for Specimen A depicted in FIGS. 11-15. As such, interface 1700 may include a combination 1750 of default text that is automatically loaded into an interface template and information selected or otherwise inputted by a technician through a series of prompts. Examples of such prompts 1730 and 1732 are provided, whereby a technician may label the specimen and enter details about the specimen (e.g., as “C2 and C3” and “Tips are submitted in cassette C1” 1760, 1770, and 1780).

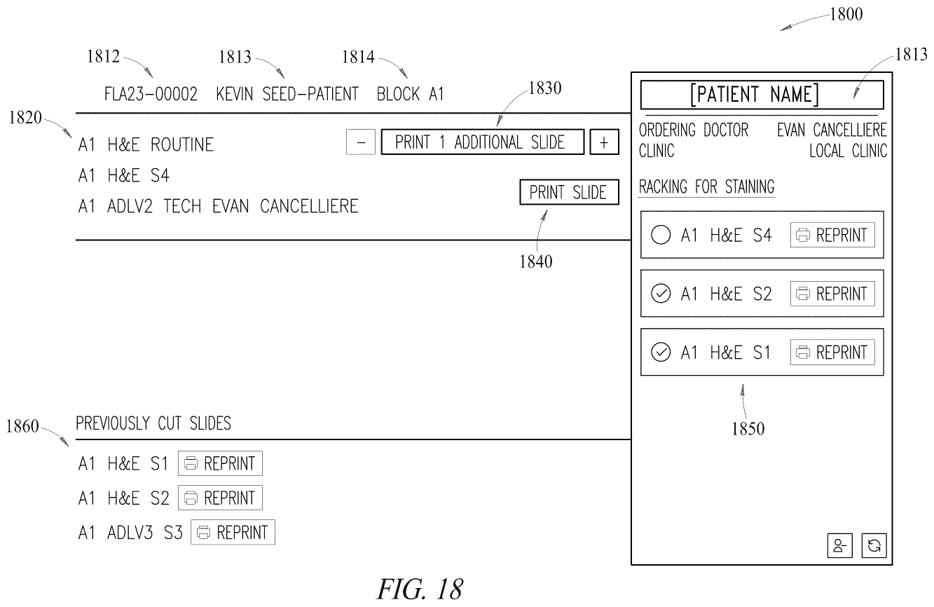

Referring to FIG. 18, interface 1800 may include an interface for printing slides. Interface 1800 may include one or more of a variety of types of information, such as a biopsy case identifier 1812, name of a patient 1813, a block identifier 1814, and an ordering doctor and receiving clinic 1813. As shown, interface 1800 may include a list of specimen identifying information 1820 (e.g., A1 H&E routine, A1 H&E S4, and A1 ADLV2 Tech), which may indicate. Here, A1 may indicate a sample or specimen identifier, H&E may indicate a staining technique that was used, “routine” may indicate that common techniques were used (e.g., the sample did not require anything out of the ordinary), S4 may represent slide 4, ADLV2 may indicate a specific type of staining that was used, and Tech may indicate the name of the technician. Interface 1800 may also include buttons 1830 and 1840 for printing a slide and/or printing additional slides. Interface 1800 may also include a status 1850 (e.g., of racking for staining and printing) of related slides (e.g., S1, S2, etc.) and a list 1860 of related slides previously cut and printed.

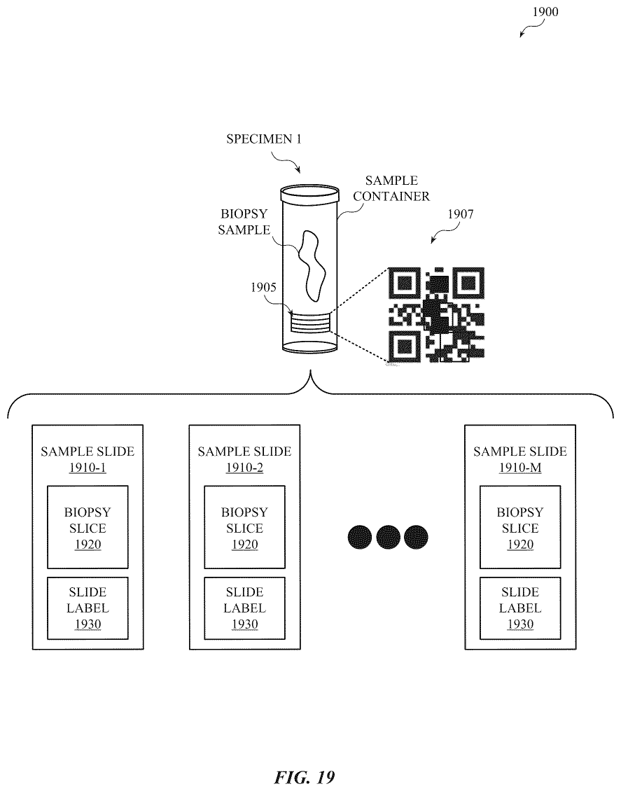

FIG. 19 is a diagram of an example 1900 of sample slides of a biopsy sample or specimen according to one or more implementations described herein. As shown, Specimen 1 may include a biopsy sample in a sample container with a specimen label. As described above with reference to FIG. 6, the specimen label 1905 may include a variety of information, such as a sample QR code 1907. As described herein, the biopsy sample may be processed to create sample slides 1910-1, 1910-2, and 1910-M (where M is greater than 2, collectively referred to as “sample slides 1910” and individually referred to as “sample slide 1910”).

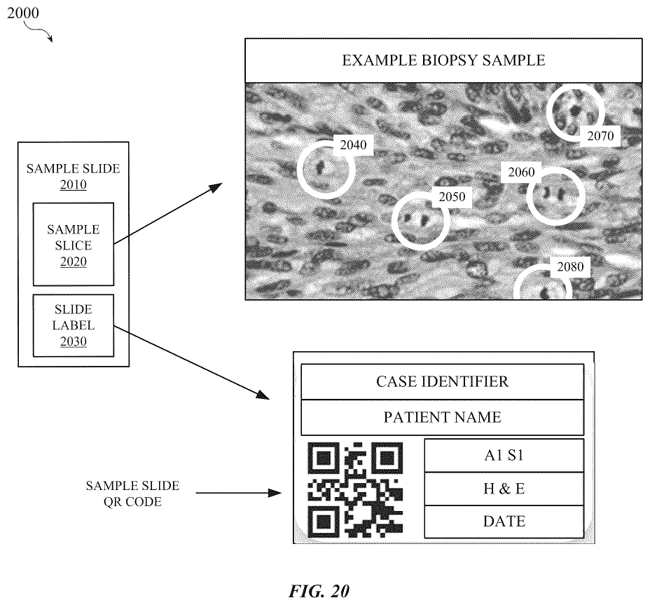

FIG. 20 is a diagram of an example 2000 of a sample slide 2010 according to one or more implementations described herein. As shown, sample slide 2010 may include biopsy slice 2020 and slide label 2030. Biopsy slice 2020 may include a thin cross-sectional slice of a corresponding biopsy sample, which may include tumors or other biological entities of interest 2040-2080. Slide label 2030 may include one or more types of information. Slide label 2030 may include a case identifier that uniquely identifies the case to which the slide corresponds, a patient name, a biopsy sample identifier (e.g., A1) and a slide identifier (e.g., S1), a . . . (e.g., H&E), a date, etc. Slide label may also include a slide QR code (or another type of scannable or visual identifier) that is associated with sample slide 2010. Slide label 2030 is provided as a non-limiting example. In some implementations, slide label 2030 may include fewer, additional, alternative, and/or alternatively arranged information than the information depicted in FIG. 20. Some or all of the information of slide label 2030 may be physically printed on the outer, protective glass (or other material) via thermal transfer and/or in another manner. Doing so may ensure that the information may not be changed.

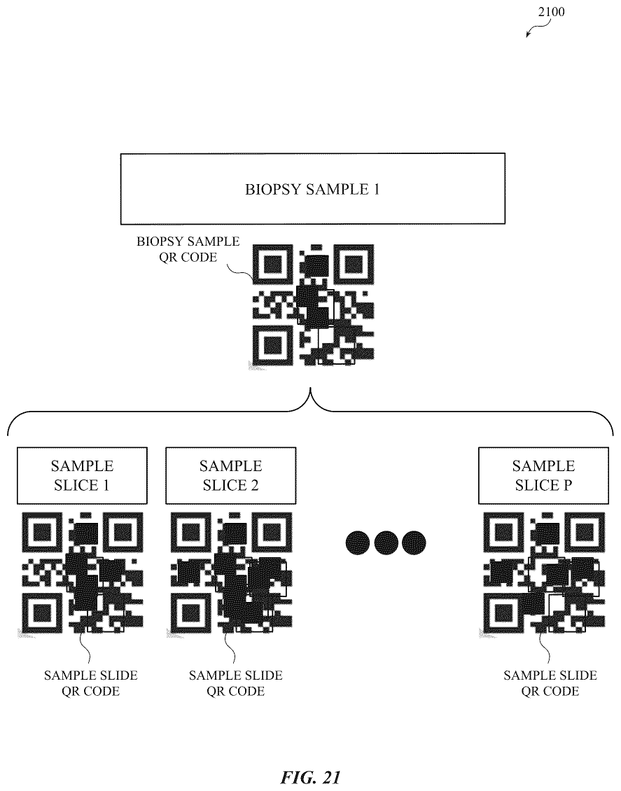

FIG. 21 is a diagram of an example 2100 of using QR codes to logically associate a biopsy sample with biopsy sample slices according to one or more implementations described herein. As shown, as a biopsy sample may be sliced into multiple sample slices, the biopsy sample and each sample slice may be assigned a QR code, and the QR code of the biopsy sample may be logically or electronically associated with the QR code of each sample slice. In this manner, as a sample slices go from one technician station 110 to another, from one laboratory to another, and/or to a doctor's office or other locations, electronic records associating the samples via QR codes may be used to access various LIS features such as tracking, record keeping, orderliness, the investigation of errors, and others.

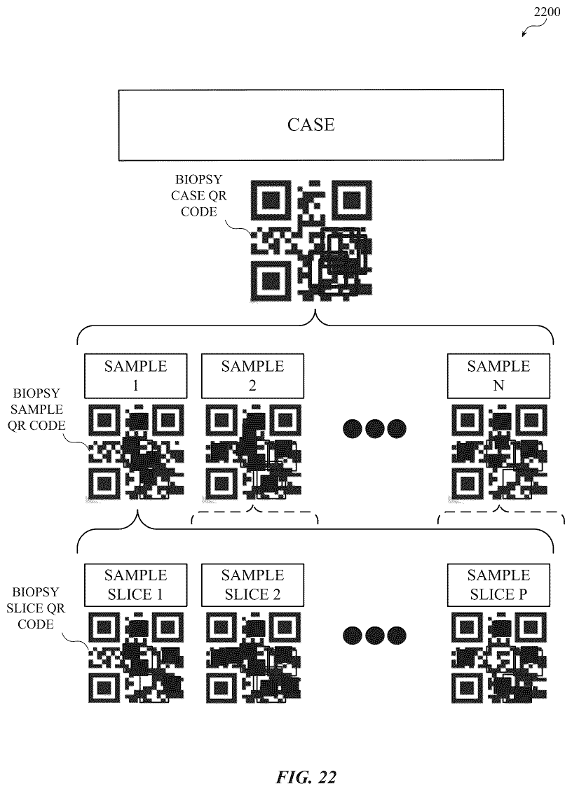

FIG. 22 is a diagram of an example of an association of QR codes between a case, biopsy samples, and sample slices according to one or more implementations described herein. FIG. 22 may be viewed as a combination of example 2100 of FIG. 21 and example 600 of FIG. 6. As shown, a case, biopsy samples, and sample slices may each be associated with a QR code (or another type of scannable identifier). The assignment and use of such a QR code scheme may greatly enhance the ability of a laboratory, team of technicians, physicians, and LIS operators to keep track of cases, samples, and slides as they are received, worked on, shipped, stored, and analyzed during the overall cancer prognosis process.

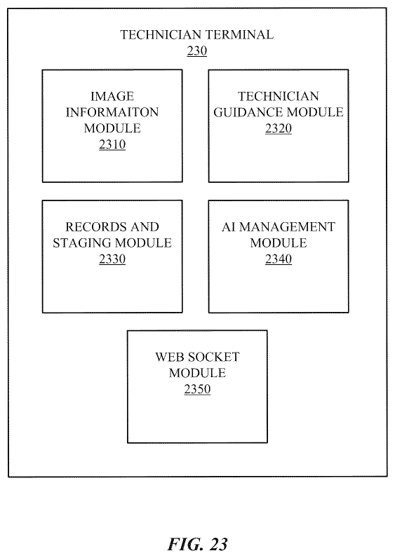

FIG. 23 is a diagram of an example 2300 of a technician terminal 230 according to one or more implementations described herein. As shown, technician terminal 230 may include image information module 2310, technician guidance module 2320, records and staging module 2330, artificial intelligence (AI) management module 2350, and web socket module 2350. The modules of FIG. 23 may represent a combination of hardware and software configured to perform at least some of the functionality of technician terminal 230. In some implementations, technician terminal 230 may include fewer, additional, alternative, or differently arranged modules (and corresponding functionality) than those depicted in FIG. 23. Additionally, or alternatively, the functionality described as being performed by one module may be performed by one or more other modules, which may or may not be depicted in FIG. 23. Further, in some implementations, one or more of the modules, and/or a function of one or more of the modules, may be implemented, in whole or in part, by another device of FIGS. 1 and/or 2, including LIS servers 120.

Image information module 2310 may be configured to receive and process input from one or more types of image or optical information. This may include one or more types of input from an image capturing device, such as scanner 210, camera 240, etc. The input may correspond to a scannable code, pattern, etc., such as a barcode, a QR code, and/or another type of optical- or image-based information scheme. The input may also, or alternatively, correspond to more general image information, such as picture and/or video information. Image information module 2310 may process the information to detect one or more patterns of interest, determine the significance of the pattern (e.g., information logically associated with the patter), and provide the information to one or more other devices or operations to facilitate or enable features described herein.

Image information module 2310 may be configured to recognize a scannable code associated with a technician ID badge, a biopsy case, a sample biopsy, and/or a sample slide. Image information module 2310 may be configured to access, retrieve, and/or share information logically associated (e.g., stored in an electronic record) with the recognized pattern. Doing so may, for example, technician terminal 230 and/or LIS server 120 to create electronic records of work being performed at different technician terminals, by different technicians, and regarding different biopsy cases, samples, and/or slides.

Image information module 2310 may also, or alternatively, be configured to generate, assign, and/or record scannable codes to a technician ID badge, a biopsy case, a sample biopsy, and/or a sample slide. For example, image information module 2310 may be used to register a technician with the LIS, generate a scannable code for the technician, and enable the technician to be recognized or registered at a technician station upon the technician's scannable code being captured by scanner 210, camera 240, etc. Additionally, or alternatively, image information module 2310 may be used to register a biopsy cases, samples, and/or slides with the LIS, such that the biopsy cases, samples, and/or slides may be detected as being at a particular technician station upon the corresponding code being captured by scanner 210, camera 240, etc.

In some implementations, image information module 2310 may be configured to implement one or more NNs to facilitate the capture, interpretation, and/or processing of image information. For example, image information module 2310 may be configured to extract a patient name using OCR technology, capture an image or video of the technician to perform facial recognition for authentication purposes, or automatically identifying attributes about the specimen, such as dimensions or type of biopsy (punch, shave, excision etc.). Neural networks may be used to identify the biopsy type, or origin on the human body that the biopsy came from. Neural networks may additionally, or alternatively, be used to detect overarching patterns in the tissue to enable technician templates to be prepopulated. Neural networks may also be used to recognize text pertaining to the patient sample. As such, image information module 2310, whether implemented by technician terminal 230, one or more LIS servers 120, and/ore a combination thereof, may support and enable a broad variety of image-based processing and analysis, whether such input may originate from scanner 210, camera 240, and/or one or more additional or alternative information sources.

Technician guidance module 2320 may be configured to determine, generate, and/or configure interfaces presented to a technician. Technician guidance module 2320 may be configured to receive an input, command, or another type of prompt that causes technician guidance module 2320 to determine an interrace template suitable for a given situation. Examples of such a prompt may include receiving scanned information from scanner 210, image information from 240, etc., indicating a biopsy case, a biopsy sample, and/or a sample slide.

Technician guidance module 2320 may also be configured to modify or customize the template interface in accordance with a current situation by, for example, adding, removing, or altering text, one or more interface objects, such as a button, a list of selectable values, phrases, or other types of inputs, etc. In some implementations, technician guidance module 2320 may select and modify a templet interface based on one or more preceding interface templates and/or inputs received from a technician via the one or more preceding interfaces. Each interface may facilitate or enable one or more functions of a technician station, such as grossing, accessing, processing, cutting, embedding, and/or digitizing a biopsy case, a biopsy sample, and/or sample slide. In some implementations, one or more NNs may be used to select or generate an interface template and/or to modify or customize the interface template in one or more ways as described herein.

Records and staging module 2330 may be configured to create, update, manage, and/or store electronic records of the LIS. In some implementations, records and staging module 2330 may be configured to register a laboratory, a physician, a technician station, a device at a technician station, a technician badge, a biopsy case, a biopsy sample, a sample slice, etc., with the LIS. Records and staging module 2330 may be configured to cause a scannable code to be generated, logically associated with a unique identifier, and an electronic record thereof to be created and stored by the LIS. Records and staging module 2330 may also, or alternatively, be configured to add, remove, and/or alter one or more attributes or other types of information associated with such records. As a biopsy case and biopsy samples are processed by technicians at technician stations records and staging module 2330 may be configured to collect information scanned, input, and otherwise generated by such events and create electronic records of the events. As such, a digital record may be created, which may be later used to address fails, lost samples, activities performed improperly or incompletely, etc.

Artificial intelligence (AI) management module 2350 may be configured to train, apply, and manage one or more types of neural networks (NNs) to one or more operations of the LIS. In some implementations, AI management module 2350 may use records created by records and staging module 2330 to train one or more NNs configured to enable the real-time assessment and evaluation of information collected from at laboratory, a technician station, a technician terminal or another technician station device, etc. This may include the identification of breaches in one or more quality standards and/or failure of one or devices or software processes being executed by one or more devices. AI management module 2350 may further be configured to use one or more NNs to analyze an error, breach in quality, or other failure and determine an appropriate remedy for the situation. Depending on the type of remedy, AI management module 2350 may generate an interface prompt to notify a technician of the break or failure and/or provide instructions to the technician. Additionally, or alternatively, AI management module 2350 may restart one or more devices, cause another device to perform an operation of a filed device, or may cancel, update, restart, or reassign to another device, the execution of one or more software operations.

Web socket module 2350 may be configured to cause or enable technician terminal 230 to communicate with one or more external systems or devices, such as LIS server 120, dermatologist system 130, courier service system 140, and system admin terminal 150. Web socket module 2350 may be configured to communicate with LIS servers 120 to receive software instructions and performance standards to be applied in the execution and evaluation of activities at technician station. Additionally, or alternatively, web socket module 2350 may be configured to communicate with LIS servers 120 to provide records or other types of information collected and processed at technician station 110. Web socket module 2350 may also, or alternatively, be configured to communicate with dermatologists to receive updates about biopsy cases and samples, instructions from physicians, and to response to requests from physicians for information and updates. Web socket module 2350 may also, or alternatively, be configured to communicate with courier service system 140 to monitor the location and travel of one or more biopsy cases, samples, slides, and/or one or other types of information. This may be facilitated as the LIS and courier service system 140 may be configured to operate by scanning the same scannable codes of biopsy cases, samples, slides, technicians, and couriers. Web socket module 2350 may also, or alternatively, be configured to communicate with a regulatory reporting system (not shown) to ensure compliance with regulatory requirements. A regulatory reporting system may include one or more servers or other computing devices capable of communicating with technician terminal 230 via a network, such as the Internet. A regulatory reporting system may be owned and operated by a regulatory body related to the generation and collection of patient and/or medical data, the use of medical equipment or devices, the handling of biopsy samples and other biological substances, etc.

Web socket module 2350 may be configured to monitor and collect information per the requirements or recommendations of one or more regulatory bodies, process the information per the requirements or recommendations of the regulatory bodies, and report the information to the regulatory bodies. Web socket module 2350 may be configured to operate at a level of granularity and according to a schedule that is consistent with that required or recommended by the regulatory bodies. Examples of information that may be provided to one or more regulatory reporting system with information may include sending information via an HL7 or FHIR interface to the state specific melanoma tracking authorities, also known as state registries. The system may also automatically or notify appropriate government officials about rare or infectious conditions, such as leprosy. The automated notification system may also notify a hospital or originating physician or dermatologist about a dangerous case, such as merkel cell, melanoma, or other condition likely to metastasize.

As such, technician terminal 230 may include one or more of a variety of modules to enable technician terminal 230 to capture and process image information in various ways, provide technicians with dynamically generated interface to help ensure accurate and efficient input information, create and manage records of what happens at technician stations 110 of a laboratory, apply NN tools to detect and dynamically remedy breaches in quality standards and device or software failures, and communicate with one or more external systems such as LIS server 120, courier service system 140, regulatory reporting systems, and more.

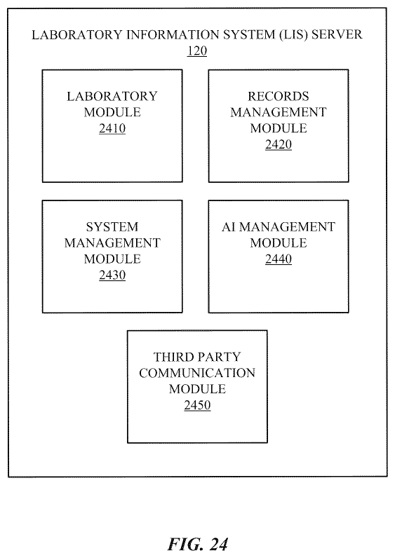

FIG. 24 is a diagram of an example LIS server 120 according to one or more implementations described herein. As shown, LIS server 120 may include laboratory module 2410, records management module 2420, system management module 2430, AI management module 2440, third party interface module 2450, and regulatory reporting module 2460. The modules of FIG. 24 may represent a combination of hardware and software configured to perform at least some of the functionality of LIS server 120. In some implementations, LIS server 120 may include fewer, additional, alternative, or differently arranged modules (and corresponding functionality) than those depicted in FIG. 24. Additionally, or alternatively, the functionality described as being performed by one module may be performed by one or more other modules, which may or may not be depicted in FIG. 24. Further, in some implementations, one or more of the modules, and/or a function of one or more of the modules, of LIS server 120 may be implemented, in whole or in part, by another device of FIGS. 1 and/or 2, including technician terminal 230.

Laboratory module 2410 may be configured to register laboratory with the LIS, which may include registering one or more technicians, biopsy cases, biopsy samples, sample slides, technician stations, and/or one or more devices of the technician stations. Laboratory module 2410 may also, or alternatively, receive status information, performance information, technician information, and one or more additional or alternative types of information from devices located at technician stations 110 at each laboratory.

Additionally, or alternatively, laboratory module 2410 may provide the collected information to records management module 2420. Based on information received, records management module 2420 may enable LIS server 120 to create organized records of technicians, laboratories, technician stations, and laboratory devices. Similarly, laboratory module 2410 may provide the collected information to system management module 2430, and system management module 2430 may be configured to monitor and evaluate the operability, efficiency, productivity of the laboratory, a technician, a technician station, and/or a device of a technician station. In some implementations, system management module 2430 may be configured to operate in conjunction with AI management module 2440 to evaluate the operability, efficiency, productivity of the laboratory, a technician, a technician station, and/or a device of a technician station. AI management module 2440 may be configured to train, apply, and manage one or more types of neural networks (NNs) to one or more operations of the LIS. In some implementations,

AI management module 2440 may use records created by records management module 2420 to train one or more NNs configured to enable the real-time assessment and evaluation of information collected from at laboratory, a technician station, a technician terminal or another technician station device, etc. This may include the identification of breaches in one or more quality standards and/or failure of one or devices or software processes being executed by one or more devices. AI management module 2440 may further be configured to use one or more NNs to analyze an error, breach in quality, or other failure and determine an appropriate remedy for the situation. Depending on the type of remedy, AI management module 2440 may generate an interface prompt to notify a technician of the break or failure and/or provide instructions to the technician, along with AI generated feedback specific to the operator, such as to use a different chemical, process, or to be more careful in handling the tissue to prevent blurriness. Additionally, or alternatively, AI management module 2440 may restart one or more devices, cause another device to perform an operation of a filed device, or may cancel, update, restart, or reassign to another device, the execution of one or more software operations.

Third-party communication module 2450 may be configured to communicate with one or more third-party systems or devices. In some implementations, third-party communication module 2450 may be configured to communicate with a courier service system to enable the geographical location of biopsy cases, biopsy samples, and biopsy slides to be tracked while in transit (e.g., between a physician's office to a laboratory). The third-party communication system may also be used to track the status of supplies (eg containers and baggies) that are shipped to various physician offices or hospitals. In such scenarios, the courier service system may use the same scannable codes as used at the laboratories and technician stations 110. In some implementations, third-party communication module 2450 may be configured to communicate with a regulatory reporting system, which may include one or more servers or other computing devices that are owned and operated by a regulatory body or organization. Third-party communication module 2450 may be configured to receive reporting requirements, schedules, and other information from the regulatory reporting system. Third-party communication module 2450 may also, or alternatively, be configured to provide information to the regulatory reporting system in accordance with the regulatory reporting requirements.

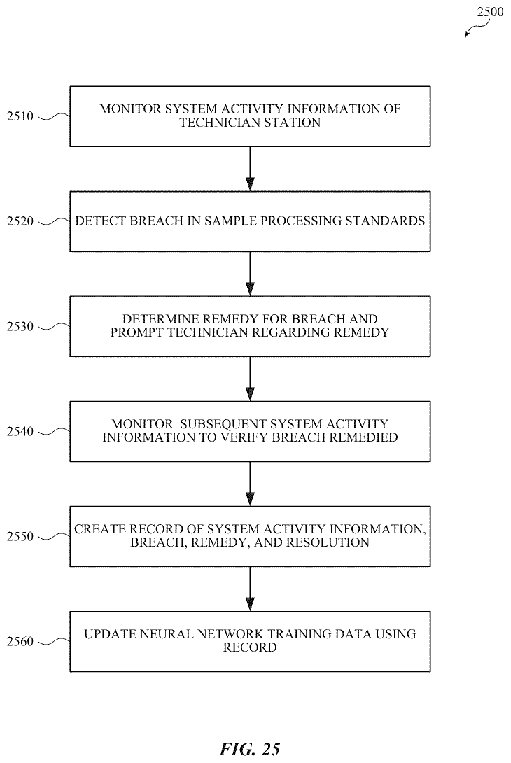

FIG. 25 is a diagram of an example of a process 2500 for dynamically ensuring technician input accuracy according to one or more implementations described herein. Process 2500 may be implemented by LIS servers 120. In some implementations, some or all of process 2500 may be performed by one or more other systems or devices, including one or more of the devices of FIG. 1 or FIG. 2, such as technician terminal 230. Additionally, process 2500 may include one or more fewer, additional, differently ordered and/or arranged operations than those shown in FIG. 25, including other processes and/or operations discussed herein. For example, process 2500 may include operations preceding, performed in parallel with, and/or following one or more of the depicted operations. Furthermore, some or all of the operations of process 2500 may be performed independently, successively, simultaneously, etc., of one or more of the other operations of process 2500. As such, the techniques described herein are not limited to a number, sequence, arrangement, timing, etc., of the operations or process depicted in FIG. 25.

As shown, process 2500 may include system activity information at technician station 110 (block 2510). For example, LIS server 120 and/or technician terminal 230 may receive image information of biopsy cases, biopsy samples, the technician working at technician station, etc. LIS server 120 and/or technician terminal 230 may also receive information indicating the operational status and functionality of one or more devices at technician station 110. LIS server 120 and/or technician terminal 230 may further receive information indicating software processes being executed or performed at technician station 110, including which software processes are being performed, whether they are performing appropriately, etc.

Process 2500 may include detecting a breach in sample processing standards (block 2520). For example, LIS server 120 and/or technician terminal 230 may detect a breach in sample processing standards based on system activity information. LIS server 120 and/or technician station 110 may apply one or more types of the monitored information (e.g., image information, device information, or software processes information) as inputs to one or more NNs configured to detect or predict breaches in sample processing standards. A sample processing standard may include one or more operational performance thresholds or patterns, information content requirements or patterns, information quality thresholds or patterns, device functionality thresholds or patterns, technical performance thresholds or patterns, one or more operational conditions, etc., including suitable procedures and metrics for assessing the same. The output of the one or more NNs may include an indication of whether a breach has occurred and/or information about the type of breach, severity of the breach, time of the breach, etc. In some implementations, LIS server 120 and/or technician terminal 230 may detect a breach in sample processing standards by comparing system activity information with sample processing standards information (e.g., without the use of a NN).

Process 2500 may include determining a remedy for the breach and prompting the technician regarding the remedy (block 2530). For example, LIS server 120 and/or technician terminal 230 may apply any combination of the monitored information (e.g., image information, device information, or software processes information) and the breach information as inputs to one or more NNs configured to determine a remedy for the breach. In some implementations, the one or more NNS used to determine the breach may also determine a suitable remedy for the breach. A suitable remedy may include one or more of: causing a notification to be provided to the technician, instructions to be provided to the technician, automatically terminating, updating, executing, re-executing, etc., one or more software processes, automatically turning on, off, or restarting a device, or causing a software process to be performed by a different device.

Process 2500 may include monitoring subsequent system activity information to verify whether the breach has been remedied (block 2540). For example, LIS server 120 and/or technician terminal 230 may receive image information of biopsy cases, biopsy samples, the technician working at technician station, etc. LIS server 120 and/or technician terminal 230 may also receive information indicating the operational status and functionality of one or more devices at technician station 110. LIS server 120 and/or technician terminal 230 may further receive information indicating software processes being executed or performed at technician station 110, including which software processes are being performed, whether they are performing appropriately, etc. LIS server 120 and/or technician terminal 230 may evaluate this information to determine whether the breach previously detected has been successfully remedied. In the event that it has not, LIS server 120 and/or technician terminal 230 may, with the remedy failure as feedback information, repeat one or more of the foregoing operations until suitable remedy is determined and implemented.

Process 2500 may include creating a record of the system activity information, the breach, remedy, and resolution (block 2550). For example, LIS server 120 and/or technician terminal 230 may create, or cause to be created, an electronic record of the system activity information, the breach, remedy, and resolution. Process 2560 may include updating NN training data using the record (block 2560). For example, LIS server 120 and/or technician terminal 230 may update NN training data using the record. In turn, LIS server 120 and/or technician terminal 230 may cause one or more NNs to be retrained using the updated training data to improve the NN's ability to detect and resolve subsequent issues.

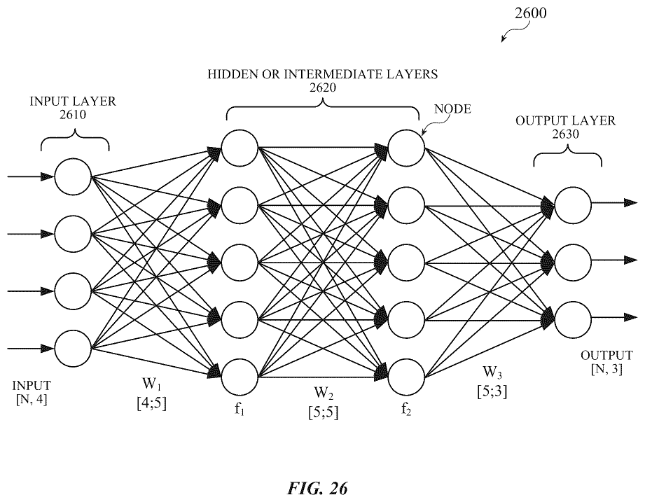

FIG. 26 is a diagram of an example of a NN 2600 according to one or more implementations described herein. As shown, NN 2600 may include nodes arranged in different layers, such as an input layer 2610, multiple hidden or intermediary layers 2620, and an output layer 2630. In some implementations, the number of nodes at the input and output layers may vary from those shown in FIG. 26. Additionally, or alternatively, the number of hidden or intermediate layers, and/or the number of nodes therein, may vary from those shown in FIG. 26.

Example NN 2600 may include a number N of inputs introduced to four input nodes [N, 26] of input layer 2610. This may include processing or encoding input data into a form, shape, vector, or data structure, that is receivable by the NN. The four input nodes may process the inputs to produce a first weighted value (W₁) that the four input nodes provide to five nodes [4;5] of a first hidden layer. The five nodes of the first hidden layer may use a first function (f₁) to process the inputs to produce a second weighted value (W₂) that the five nodes of the first hidden layer may provide to the five nodes [5;5] of a second hidden layer. The five nodes of the second layer may use a second function (f₂) to process the inputs to produce a third weight (W₃) that the five nodes of the second hidden layer may provide to the three nodes [5;3] of output layer 2630. The nodes of output layer 2630 may each process the inputs received and produce an output. This may include converting or unencoding output data from a form, shape, vector, or data structure, that may be used by a subsequent algorithm, process, or procedure.

As described herein, the techniques described herein may include an integrated NN. In some implementations, the learned features of multiple NNs may be combined to form an integrated NN, by encoding the outputs from other NNs and introducing the encoded outputs into a hidden or intermediate layer of the integrated NN. For example, in some implementations, the integrated NN may be an image analysis NN that is modified or augmented by the incorporation of encoded outputs of other (non-image) NNs.

Artificial intelligence (AI) may involve the combination of computer science and datasets to enable problem-solving. AI may encompass machine learning (ML) and deep learning (DL). These disciplines may comprise of AI or NN algorithms designed to create expert systems which make predictions or classifications based on input data. NNs may be a type of DL algorithm, and DL may be a sub-field of ML. DL and ML algorithms may differ in how each type of algorithm learns. Deep ML may use labeled datasets (also known as supervised learning) to inform its algorithm, but Deep ML does not necessarily require a labeled dataset. DL may ingest unstructured data in its raw form (e.g., text or images) and may automatically determine the set of features which distinguish different categories of data from one another. This may eliminate some of the human intervention required and enable use of larger data sets.

NNs or artificial NNs (ANNs) may comprise logically interconnected nodes arranged in node layers. There may be an input layer, one or more hidden or intermediate layers, and an output layer. Each node, or artificial neuron, may connect to another and has an associated weight and threshold. If the output of any individual node is above the specified threshold value, that node may be activated, sending data to the next layer of the network. Otherwise, no data may be passed along to the next layer of the network by that node. The “deep” in DL may refer to the number of layers in a NN. A NN that consists of more than three layers—which would be inclusive of the input and the output—can be considered a deep learning algorithm or a deep NN.

Feedforward NNs may include an input layer, one or more hidden layers, and an output layer. One type of feedforward NN, referred to as a multi-layer perceptron (MLP), may be one where all, or multiple, layers of the NN are fully connected, meaning that each layer that feeds into the next layer connects to all nodes in the next layer. These networks may learn from input data and may function as a foundation for computer vision, natural language processing, and other neural networks. Recurrent neural networks (RNNs) may be identified by feedback loops. Convolutional NNs (CNNs) may be similar to MLPs, but they differ in that not all layers are fully connected and instead have convolutional layers and pooling layers and may be used for image recognition, pattern recognition, and/or computer vision.

NNs may harness principles from linear algebra, particularly matrix multiplication, to identify patterns within an image. Linear regression analysis, for example, may be used to predict a value of a variable based on a value of another variable. This form of analysis may estimate coefficients of a linear equation, involving one or more independent variables that best predict the value of the dependent variable. Linear regression may fit a straight line or surface that minimizes discrepancies between a predicted value and an actual value. These learning algorithms may be leveraged when using time-series data to make predictions about future outcomes.

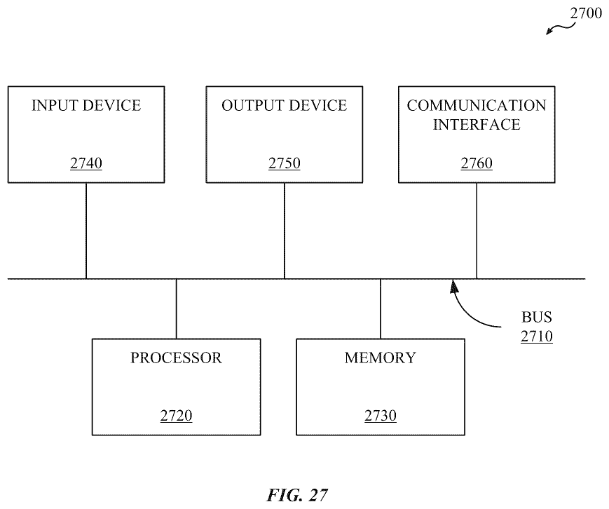

FIG. 27 is a diagram of example components of a device 2700 that may be used within environment 100 of FIG. 1 or devices of FIG. 2. Device 2700 may correspond to technician station 110, LIS servers 120, dermatologist system 130, courier service system 140, system admin terminal 150. Each of technician station 110, LIS servers 120, dermatologist system 130, courier service system 140, system admin terminal 150 may include one or more of devices 2700 and/or one or more of the components of device 2700. Device 2700 may also, or alternatively, correspond to scanner 210, technician terminal 220, station-specific devices 230, camera 240, and/or scannable code device and printer 250. One or more of the devices described herein may include one or more fewer, additional, alternative, and/or alternatively arranged components relative to those depicted in FIG. 27.

As depicted, device 2700 may include bus 2710, processor 2720, memory 2730, input device 2740, output device 2750, and communication interface 2760. However, the precise components of device 2700 may vary between implementations. For example, depending on the implementation, device 2700 may include fewer components, additional components, different components, or differently arranged components than those illustrated in FIG. 27.

Bus 2710 may permit communication among the components of device 2700.

Processor 2720 may include one or more processors, microprocessors, data processors, co-processors, network processors, application-specific integrated circuits (ASICs), controllers, programmable logic devices (PLDs), chipsets, field-programmable gate arrays (FPGAs), or other components that may interpret or execute instructions or data. Processor 2720 may control the overall operation, or a portion thereof, of device 2700, based on, for example, an operating system (not illustrated), and/or various applications. Processor 2720 may access instructions from memory 2730, from other components of device 2700, or from a source external to device 2700 (e.g., a network or another device).

Memory 2730 may include memory and/or secondary storage. For example, memory 2730 may include random access memory (RAM), dynamic RAM (DRAM), read-only memory (ROM), programmable ROM (PROM), flash memory, or some other type of memory. Memory 2730 may include a hard disk (e.g., a magnetic disk, an optical disk, a magneto-optic disk, a solid state disk, etc.) or some other type of computer-readable medium, along with a corresponding drive. A computer-readable medium may be defined as a non-transitory memory device. A memory device may include space within a single physical memory device or spread across multiple physical memory devices.

Input device 2740 may include one or more components that permit a user to input information into device 2700. For example, input device 2740 may include a keypad, a button, a switch, a knob, fingerprint recognition logic, retinal scan logic, a web cam, voice recognition logic, a touchpad, an input port, a microphone, a display, or some other type of input component. Output device 2750 may include one or more components that permit device 2700 to output information to a user. For example, output device 2750 may include a display, light-emitting diodes (LEDs), an output port, a speaker, or some other type of output component.

Communication interface 2760 may include one or more components that permit device 2700 to communicate with other devices or networks. For example, communication interface 2760 may include some type of wireless or wired interface. Communication interface 2760 may also include an antenna (or a set of antennas) that permit wireless communication, such as the transmission and reception of radio frequency (RF) signals.

As described herein, device 2700 may perform certain operations in response to processor 2720 executing software instructions contained in a computer-readable medium, such as memory 2730. The software instructions may be read into memory 2730 from another computer-readable medium or from another device via communication interface 2760. The software instructions contained in memory 2730 may cause processor 2720 to perform one or more processes described herein. Alternatively, hardwired circuitry may be used in place of, or in combination with, software instructions to implement processes described herein. Thus, implementations described herein are not limited to any specific combination of hardware circuitry and software.

Examples herein can include subject matter such as a method, means for performing acts or blocks of the method, at least one machine-readable medium including executable instructions that, when performed by a machine (e.g., a processor (e.g., processor, etc.) with memory, an application-specific integrated circuit (ASIC), a field programmable gate array (FPGA), or the like) cause the machine to perform acts of the method or of an apparatus or system for concurrent communication using multiple communication technologies according to implementations and examples described.

In example 1, which may also include one or more of the examples described herein, a device, comprising: a memory configured to store instructions; and one or more processors configured to, when executing the instructions stored in the memory, cause the server device to: receive scanned sample information corresponding to a biopsy sample; dynamically determine, based on the scanned information, an interface of a series of interfaces for inputting information about the biopsy sample while processing the biopsy sample at a technician station, the interface comprising template information comprising an incomplete input data, one or more prompts for entering information to satisfy the incomplete input data, and one or more selectable interface objects comprising pre-generated values for satisfying the one or more prompts; cause the interface to be presented via a technician terminal; detect a selection of a selectable interface object of the one or more selectable interface objects relative to the a prompt of the one or more prompts; and update the incomplete input data of the interface to comprise the pre-generated value of the selectable interface object.

In example 2, which may also include one or more of the examples described herein, the device is the technician terminal of a technician station, the technician terminal.

In example 3, which may also include one or more of the examples described herein, the technician station comprises one or more station-specific devices configured to enable at least one of: accessioning the biopsy sample, grossing the biopsy sample, processing the biopsy sample, cutting and embedding the biopsy sample, staining the biopsy sample, or digitizing the biopsy sample.

In example 4, which may also include one or more of the examples described herein, the technician station comprises: the technician terminal; the one or more station-specific devices; and at least one of: a scanner device; a camera configured to capture image information at the technician station; a scannable code device configured to determine scannable codes for biopsy cases, biopsy samples, or sample slices; or a printer configured to print the scannable codes.

In example 5, which may also include one or more of the examples described herein, the device is a laboratory information system (LIS) server device.

In example 6, which may also include one or more of the examples described herein, the scanned information originates from a scanner device of a technician station.

In example 7, which may also include one or more of the examples described herein, the scanned information originates from a camera of a technician station.

In example 8, which may also include one or more of the examples described herein, the one or more processors are further configured to: dynamically determine a subsequent interface, of the series of interfaces, based on a combination of the scanned information and information received via one or more prior interfaces.

In example 9, which may also include one or more of the examples described herein, the subsequent interface is determined by: determining a template interface for the subsequent interface; and modifying the template interface for the subsequent interface based on the combination of the scanned information and the information received via one or more prior interfaces.

In example 10, which may also include one or more of the examples described herein, the template interface for the subsequent interface is modified by at least one of: adding, removing, or modifying a pressable button; adding, removing, or modifying text; adding, removing, or modifying an interface prompt directed to the technician; adding, removing, or modifying one or more pre-generated phrases configured to be selectable as input; or adding, removing, or modifying one or more values configured to be selectable as input.

In example 11, which may also include one or more of the examples described herein, the subsequent interface is based on a corresponding template.

In example 12, which may also include one or more of the examples described herein, the one or more processors are further configured to: cause an electronic record, corresponding to the biopsy sample, to be created or updated based on the information input via the interface.

In example 13, which may also include one or more of the examples described herein, the interface comprises at least one of the following interface objects: a case identifier configured to uniquely identify a biopsy case to which the biopsy sample corresponds; a sample identifier configured to uniquely identify the biopsy sample; a name of a physician associated with the biopsy; a name of a clinic associated with the biopsy; a patient name; clinical notes regarding the biopsy case or biopsy sample; historical information about the biopsy case or biopsy sample; a name of a technician working on the biopsy sample; a button to enable text to be included in the interface; one or more pre-generated phrases describing a condition or feature of the biopsy sample, the one or more pre-generated phrases being configured to be included in the interfaces upon selection; or a button to cause at least one cassettes to be printed.

In example 14, which may also include one or more of the examples described herein, the one or more processors are further configured to: cause a scannable code to be determined for each sample slice of a plurality of sample slices of the biopsy sample; and cause at least one electronic record to be updated or created to logically associate the scannable code for each sample slice to a scannable code of the biopsy sample.

In example 15, which may also include one or more of the examples described herein, the one or more processors are further configured to: cause at least one electronic record to be updated or created to logically associate a scannable code that uniquely identifies the biopsy sample to a scannable code that uniquely identifies a corresponding biopsy case.

In example 16, which may also include one or more of the examples described herein, a method, performed by a device, comprising: receiving scanned sample information corresponding to a biopsy sample; dynamically determining, based on the scanned information, an interface of a series of interfaces for inputting information about the biopsy sample while processing the biopsy sample at a technician station, the interface comprising template information comprising an incomplete input data, one or more prompts for entering information to satisfy the incomplete input data, and one or more selectable interface objects comprising pre-generated values for satisfying the one or more prompts; causing the interface to be presented via a technician terminal; detecting a selection of a selectable interface object of the one or more selectable interface objects relative to the a prompt of the one or more prompts; and updating the incomplete input data of the interface to comprise the pre-generated value of the selectable interface object.

In example 17, which may also include one or more of the examples described herein, a non-transitory, computer-readable medium comprising: one or more instructions that when executed by one or more processors cause the one or more processors to: receive scanned sample information corresponding to a biopsy sample; dynamically determine, based on the scanned information, an interface of a series of interfaces for inputting information about the biopsy sample while processing the biopsy sample at a technician station, the interface comprising template information comprising an incomplete input data, one or more prompts for entering information to satisfy the incomplete input data, and one or more selectable interface objects comprising pre-generated values for satisfying the one or more prompts; cause the interface to be presented via a technician terminal; detect a selection of a selectable interface object of the one or more selectable interface objects relative to the a prompt of the one or more prompts; and update the incomplete input data of the interface to comprise the pre-generated value of the selectable interface object.

In example 18, which may also include one or more of the examples described herein, a device, comprising: a memory configured to store instructions; and one or more processors configured to, when executing the instructions stored in the memory, cause the server device to: receive system activity information corresponding to a technician station; monitor the system activity information to detect a breach in sample processing standards; apply the system activity information and an indication of the breach as input information to one or more neural networks (NNs), the NNs being trained using historical system activity information, historical breaches in sample processing standards, and suitable remedies for the historical breaches; determine, based on an output of the one or more NNs, an appropriate remedy for the breach; execute the appropriate remedy for the breach; and verify, based on additional system activity information received after execution of the appropriate remedy, that the breach has been remedied.

In example 19, which may also include one or more of the examples described herein, the sample processing standards comprise at least one of: one or more operational performance thresholds; one or more operational performance patterns; one or more information content requirements; one or more information content patterns; one or more information quality thresholds; one or more information quality patterns; one or more device functionality thresholds; one or more device functionality patterns; one or more technical performance thresholds; one or more technical performance patterns; or one or more operational conditions. A simple detector, not built on a neural network, may be used to catch abnormalities, or to detect that there is a process out of tolerance. For example, detecting a color set using RGB values that is not close to the average color set for other slides.

In example 20, which may also include one or more of the examples described herein, the system activity information comprises at least one of: information associated with technician conduct, information associated with a performance of one or more devices, or information associated with a performance of one or more software processes.

In example 21, which may also include one or more of the examples described herein, detecting a breach may include determining that the system activity information does not comply with one or more sample processing standards.

In example 22, which may also include one or more of the examples described herein, the appropriate remedy comprises at least one of: notifying a technician of the breach, providing the technician with one or more instructions regarding the breach, pausing, canceling, undoing, or redoing one or more device operations, dynamically altering or updating one or more device operations, dynamically updating one or more electronic records, causing one or more device operations to be performed by one or more other devices, creating a record of the system activity information, the breach, and the remedy.

In example 23, which may also include one or more of the examples described herein, the one or more processors may be configured to: monitor the monitor the system activity information to detect a breach in sample processing standards by: applying the system activity information as input to a sample processing standards NN, the sample processing standards NN being trained on historical system activity information, historical breaches in sample processing standards, and historical sources of breaches in sample processing standards, and the sample processing standards NN being configured to provide an indication of whether one or more aspects of the system activity information amounts to one or more breaches in sample processing standards. Note that a breach might also, or alternatively, be a data breach, including a technician sharing a login or protected health information (PHI) data. In such scenarios, the system may be configured to detect a copy/paste command, information being extracted from the system, etc.

In example 24, which may also include one or more of the examples described herein, the device comprises at least one of: the technician terminal, one or more laboratory information system (LIS) servers, a combination of the technician terminal and the one or more laboratory information system (LIS) servers.

In example 25, which may also include one or more of the examples described herein, the device further comprises at least one of: a scanner device; a camera configured to capture image information at the technician station; a scannable code device configured to determine scannable codes for biopsy cases, biopsy samples, or sample slices; or a printer configured to print the scannable codes.

In example 26, which may also include one or more of the examples described herein, the technician performance information comprises at least one of: video information captured by a camera regarding an action or sequence of actions performed by the technician at the technician station, an input or a sequence of inputs, by the technician, into one or more of the device of the technician station, or an operation or sequence of operations performed by one or more of the device of the technician station. If the sequence detected by video is out of order, or an incorrect motion is detected, real time correction or suggestions may also be presented to the technician. This may include using neural networks to recognize human movement patterns, movements out of tolerance, etc.

In example 27, which may also include one or more of the examples described herein, the technician performance information comprises at least one of: creating a scannable code that uniquely identifies a biopsy case, scanning the scannable code that uniquely identifies a biopsy case, creating a scannable code that uniquely identifies a biopsy sample, scanning the scannable code that uniquely identifies a biopsy sample, creating a scannable code that uniquely identifies a sample slide, or scanning the scannable code that uniquely identifies a sample slide.

In example 28, which may also include one or more of the examples described herein, the technician performance information corresponds to information relating to the technician performing at least one of the following: accessioning the biopsy sample, grossing the biopsy sample, processing the biopsy sample, cutting and embedding the biopsy sample, staining the biopsy sample, or digitizing the biopsy sample.

In example 29, which may also include one or more of the examples described herein, the one or more processors are further configured to: create an electronic record of the technician performance information, the breach, the remedy, and whether the breach was remedied.

In example 30, which may also include one or more of the examples described herein, the record further comprises at least one of: a technician identifier that uniquely identifies the technician, a technician station identifier that uniquely identifies the technician station, a technician terminal identifier that uniquely identifies the technician terminal, a unique identifier of another device at the technician station, or a time and/or date of the breach.

In example 31, which may also include one or more of the examples described herein, the one or more processors are further configured to: create an updated set of NN training data using the electronic record and a set of NN training data; and train the one or more NNs using the updated set of NN training data.

In example 32, which may also include one or more of the examples described herein, the one or more processors are further configured to: use the one or more NNs to monitor subsequent technician performance information for patterns or conditions similar to those leading to the breach.

In example 33, which may also include one or more of the examples described herein, a method, performed by a device, comprising: receiving technician performance information corresponding to a technician at a technician station; monitoring the technician performance information to detect a breach in sample processing standards; applying the technician performance information and an indication of the breach as input information to one or more neural networks (NNs), the NNs being trained using historical technician performance information, historical breaches in sample processing standards, and suitable remedies for the historical breaches; determining, based on an output of the one or more NNs, an appropriate remedy for the breach; dynamically generating a prompt comprising an indication of the breach and the remedy and cause a technician terminal, at the technician station, to generate the prompt; and verifying, based on additional technician performance information received after generating the prompt, that the breach has been remedied.

In example 34, which may also include one or more of the examples described herein, a non-transitory, computer-readable medium comprising: one or more instructions that when executed by one or more processors cause the one or more processors to: receive technician performance information corresponding to a technician at a technician station; monitor the technician performance information to detect a breach in sample processing standards; apply the technician performance information and an indication of the breach as input information to one or more neural networks (NNs), the NNs being trained using historical technician performance information, historical breaches in sample processing standards, and suitable remedies for the historical breaches; determine, based on an output of the one or more NNs, an appropriate remedy for the breach; dynamically generate a prompt comprising an indication of the breach and the remedy and cause a technician terminal, at the technician station, to generate the prompt; and verify, based on additional technician performance information received after generating the prompt, that the breach has been remedied.

The above description of illustrated examples, implementations, aspects, etc., of the subject disclosure, including what is described in the Abstract, is not intended to be exhaustive or to limit the disclosed aspects to the precise forms disclosed. While specific examples, implementations, aspects, etc., are described herein for illustrative purposes, various modifications are possible that are considered within the scope of such examples, implementations, aspects, etc., as those skilled in the relevant art can recognize.

In this regard, while the disclosed subject matter has been described in connection with various examples, implementations, aspects, etc., and corresponding Figures, where applicable, it is to be understood that other similar aspects can be used or modifications and additions can be made to the disclosed subject matter for performing the same, similar, alternative, or substitute function of the subject matter without deviating therefrom. Therefore, the disclosed subject matter should not be limited to any single example, implementation, or aspect described herein, but rather should be construed in breadth and scope in accordance with the appended claims below.

In particular regard to the various functions performed by the above described components or structures (assemblies, devices, circuits, systems, etc.), the terms (including a reference to a “means”) used to describe such components are intended to correspond, unless otherwise indicated, to any component or structure which performs the specified function of the described component (e.g., that is functionally equivalent), even though not structurally equivalent to the disclosed structure which performs the function in the herein illustrated exemplary implementations. In addition, while a particular feature may have been disclosed with respect to only one of several implementations, such feature may be combined with one or more other features of the other implementations as may be desired and advantageous for any given application.

As used herein, the term “or” is intended to mean an inclusive “or” rather than an exclusive “or”. That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more” unless specified otherwise or clear from context to be directed to a singular form. Furthermore, to the extent that the terms “including”, “includes”, “having”, “has”, “with”, or variants thereof are used in either the detailed description or the claims, such terms are intended to be inclusive in a manner similar to the term “comprising.” Additionally, in situations wherein one or more numbered items are discussed (e.g., a “first X”, a “second X”, etc.), in general the one or more numbered items can be distinct, or they can be the same, although in some situations the context may indicate that they are distinct or that they are the same.

It is well understood that the use of personally identifiable information should follow privacy policies and practices that are generally recognized as meeting or exceeding industry or governmental requirements for maintaining the privacy of users. In particular, personally identifiable information data should be managed and handled to minimize risks of unintentional or unauthorized access or use, and the nature of authorized use should be clearly indicated to users.

1. A device, comprising:
a memory configured to store instructions; and
one or more processors configured to, when executing the instructions stored in the memory, cause the server device to:
receive scanned sample information corresponding to a biopsy sample;
dynamically determine, based on the scanned information, an interface of a series of interfaces for inputting information about the biopsy sample while performing tissue processing on the biopsy sample at a technician station, the interface comprising template information comprising an incomplete input data, one or more prompts for entering information to satisfy the incomplete input data, and one or more selectable interface objects comprising pre-generated values for satisfying the one or more prompts;
cause the interface to be presented via a technician terminal;
detect a selection of a selectable interface object of the one or more selectable interface objects relative to the a prompt of the one or more prompts; and
update the incomplete input data of the interface to comprise the pre-generated value of the selectable interface object.

2. The device of claim 1, wherein the device is the technician terminal of a technician station, the technician terminal.

3. The device of claim 2, wherein the technician station comprises one or more station-specific devices configured to enable at least one of:
accessioning the biopsy sample,
grossing the biopsy sample,
performing tissue processing on the biopsy sample,
cutting and embedding the biopsy sample,
staining the biopsy sample, or
digitizing the slide resulting from the biopsy sample.

4. The device of claim 3, wherein the technician station comprises:
the technician terminal;
the one or more station-specific devices; and
at least one of:
a scanner device,
a camera configured to capture image or video information at the technician station,
a scannable code device configured to determine scannable codes for biopsy cases, biopsy samples, or sample slices, or
a printer configured to print the scannable codes.

5. The device of claim 1, wherein the device is a laboratory information system (LIS) server device.

6. The device of claim 1, wherein the scanned information originates from a scanner device of a technician station.

7. The device of claim 1, wherein the scanned information originates from a camera of a technician station.

8. The device of claim 1, wherein the one or more processors are further configured to:
dynamically determine a subsequent interface, of the series of interfaces, based on a combination of the scanned information and information received via one or more prior interfaces.

9. The device of claim 8, wherein the subsequent interface is determined by:
determining a template interface for the subsequent interface; and
modifying the template interface for the subsequent interface based on the combination of the scanned information and the information received via one or more prior interfaces.

10. The device of claim 9, wherein the template interface for the subsequent interface is modified by at least one of:
adding, removing, or modifying a pressable button,
adding, removing, or modifying text,
adding, removing, or modifying an interface prompt directed to the technician,
adding, removing, or modifying one or more pre-generated phrases configured to be selectable as input,
adding, removing, or modifying one or more values configured to be selectable as input, or
adding, removing, or modifying free text form fields

11. The device of claim 10, wherein the subsequent interface is based on a corresponding template.

12. The device of claim 1, wherein the one or more processors are further configured to:
cause an electronic record, corresponding to the biopsy sample, to be created or updated based on the information input via the interface.

13. The device of claim 1, wherein the interface comprises at least one of the following interface objects:
a case identifier configured to uniquely identify a biopsy case to which the biopsy sample corresponds,
a sample identifier configured to uniquely identify the biopsy sample,
a name of a physician associated with the biopsy,
a name of a clinic associated with the biopsy,
a patient name,
clinical notes regarding the biopsy case or biopsy sample,
historical information about the biopsy case or biopsy sample,
a name or identifier of a technician working on the biopsy sample,
a button to enable text to be included in the interface,
one or more pre-generated phrases describing a condition or feature of the biopsy sample, the one or more pre-generated phrases being configured to be included in the interfaces upon selection, or
a button to cause at least one cassette label to be printed.

14. The device of claim 1, wherein the one or more processors are further configured to:
cause a scannable code to be determined for each sample slice of a plurality of sample slices of the biopsy sample; and
cause at least one electronic record to be updated or created to logically associate the scannable code for each sample slice to a scannable code of the biopsy sample.

15. The device of claim 1, wherein the one or more processors are further configured to:
cause at least one electronic record to be updated or created to logically associate a scannable code that uniquely identifies the biopsy sample to a scannable code that uniquely identifies a corresponding biopsy case.

16. A method, performed by a device, comprising:
receiving scanned sample information corresponding to a biopsy sample;
dynamically determining, based on the scanned information, an interface of a series of interfaces for inputting information about the biopsy sample while tissue processing the biopsy sample at a technician station, the interface comprising template information comprising an incomplete input data, one or more prompts for entering information to satisfy the incomplete input data, and one or more selectable interface objects comprising pre-generated values for satisfying the one or more prompts;
causing the interface to be presented via a technician terminal;
detecting a selection of a selectable interface object of the one or more selectable interface objects relative to the a prompt of the one or more prompts; and
updating the incomplete input data of the interface to comprise the pre-generated value of the selectable interface object.

17. The method of claim 16, wherein the device is the technician terminal of a technician station, the technician terminal.

18. The method of claim 17, wherein the technician station comprises one or more station-specific devices configured to enable at least one of:
accessioning the biopsy sample,
grossing the biopsy sample,
tissue processing of the biopsy sample,
cutting and embedding the biopsy sample,
staining the biopsy sample, or
digitizing the biopsy sample.

19. The method of claim 18, wherein the technician station comprises:
the technician terminal,
the one or more station-specific devices, and
at least one of:
a scanner device,
a camera configured to capture image information at the technician station
a scannable code device configured to determine scannable codes for biopsy cases, biopsy samples, or sample slices; or
a printer configured to print the scannable codes.

20. The method of claim 16, wherein the device is a laboratory information system (LIS) server device.

21. The method of claim 16, wherein the scanned information originates from a scanner device of a technician station.

22. The method of claim 16, wherein the scanned information originates from a camera of a technician station.

23. The method of claim 16, further comprising:
dynamically determining a subsequent interface, of the series of interfaces, based on a combination of the scanned information and information received via one or more prior interfaces.

24. The method of claim 16, further comprising:
causing an electronic record, corresponding to the biopsy sample, to be created or updated based on the information input via the interface.

25. The method of claim 16, wherein the interface comprises at least one of the following interface objects:
a case identifier configured to uniquely identify a biopsy case to which the biopsy sample corresponds,
a sample identifier configured to uniquely identify the biopsy sample,
a name of a physician associated with the biopsy,
a name of a clinic associated with the biopsy,
a patient name,
clinical notes regarding the biopsy case or biopsy sample,
historical information about the biopsy case or biopsy sample,
a name of a technician working on the biopsy sample,
a button to enable text to be included in the interface,
one or more pre-generated phrases describing a condition or feature of the biopsy sample, the one or more pre-generated phrases being configured to be included in the interfaces upon selection, or
a button to cause at least one cassette label to be printed.

26. The method of claim 16, further comprising:
causing a scannable code to be determined for each sample slice of a plurality of sample slices of the biopsy sample; and
causing at least one electronic record to be updated or created to logically associate the scannable code for each sample slice to a scannable code of the biopsy sample.

27. The method of claim 16, further comprising:
causing at least one electronic record to be updated or created to logically associate a scannable code that uniquely identifies the biopsy sample to a scannable code that uniquely identifies a corresponding biopsy case.

28. A non-transitory, computer-readable medium comprising:
one or more instructions that when executed by one or more processors cause the one or more processors to:
receive scanned sample information corresponding to a biopsy sample;
dynamically determine, based on the scanned information, an interface of a series of interfaces for inputting information about the biopsy sample while tissue processing the biopsy sample at a technician station, the interface comprising template information comprising an incomplete input data, one or more prompts for entering information to satisfy the incomplete input data, and one or more selectable interface objects comprising pre-generated values for satisfying the one or more prompts;
cause the interface to be presented via a technician terminal;
detect a selection of a selectable interface object of the one or more selectable interface objects relative to the a prompt of the one or more prompts; and
update the incomplete input data of the interface to comprise the pre-generated value of the selectable interface object.

29. The non-transitory, computer-readable medium of claim 28, wherein the one or more processors are further to:
dynamically determine a subsequent interface, of the series of interfaces, based on a combination of the scanned information and information received via one or more prior interfaces.

30. The non-transitory, computer-readable medium of claim 28, wherein the one or more processors are further to:
cause an electronic record, corresponding to the biopsy sample, to be created or updated based on the information input via the interface.

31. The non-transitory, computer-readable medium of claim 28, wherein the interface comprises at least one of the following interface objects:
a case identifier configured to uniquely identify a biopsy case to which the biopsy sample corresponds,
a sample identifier configured to uniquely identify the biopsy sample,
a name of a physician associated with the biopsy,
a name of a clinic associated with the biopsy,
a patient name,
clinical notes regarding the biopsy case or biopsy sample,
historical information about the biopsy case or biopsy sample,
a name of a technician working on the biopsy sample,
a button to enable text to be included in the interface,
one or more pre-generated phrases describing a condition or feature of the biopsy sample, the one or more pre-generated phrases being configured to be included in the interfaces upon selection, or
a button to cause at least one cassette label to be printed.

32. The non-transitory, computer-readable medium of claim 28, wherein the one or more processors are further to:
cause a scannable code to be determined for each sample slice of a plurality of sample slices of the biopsy sample; and
cause at least one electronic record to be updated or created to logically associate the scannable code for each sample slice to a scannable code of the biopsy sample.

33. The non-transitory, computer-readable medium of claim 28, wherein the one or more processors are further to:
cause at least one electronic record to be updated or created to logically associate a scannable code that uniquely identifies the biopsy sample to a scannable code that uniquely identifies a corresponding biopsy case.
